(19) United States
(12) Patent Application Publication
(10) Pub. No.: US 2017/0333493 A1
(43) Pub. Date: Nov. 23, 2017

(54) PROBIOTIC BACTERIA FOR THE PREVENTION AND TREATMENT OF SALMONELLA

(71) Applicant: Ohio State Innovation Foundation, Columbus, OH (US)

(72) Inventors: Brian AHMER, Lewis Center, OH (US); Anice SABAG-DAIGLE, Columbus, OH (US)

(21) Appl. No.: 15/525,867

(22) PCT Filed: Nov. 11, 2015
(86) PCT No.: PCT/US2015/060141

§ 371 Date: May 10, 2017

Related U.S. Application Data
(60) Provisional application No. 62/078,100, filed on Nov. 11, 2014.

(51) Int. Cl. A61K 35/741; A61K 9/06; A61K 9/14; A61K 35/00

(52) U.S. Cl. CPC A61K 35/741; A61K 9/14; A61K 9/06; A61K 2035/11

(57) ABSTRACT

As disclosed herein, fructose-asparagine (F-Asn) is a primary nutrient utilized during Salmonella-mediated gastroenteritis. Engineered bacteria are disclosed that can compete with Salmonella for F-Asn and other nutrients and withstand Salmonella-induced inflammation. These bacterium can be used as probiotics to treat and prevent Salmonella-mediated gastroenteritis.

### CROSS-REFERENCE TO RELATED APPLICATIONS

This application claims benefit of U.S. Provisional Application No. 62/078,100, filed Nov. 11, 2014, which is hereby incorporated herein by reference in its entirety.

### STATEMENT REGARDING FEDERALLY SPONSORED RESEARCH OR DEVELOPMENT

This invention was made with Government Support under Grant Nos. AI073971 and AI097116, and AI116119, awarded by the National Institutes of Health. The Government has certain rights in the invention.

### BACKGROUND

Salmonella is a foodborne pathogen that causes significant morbidity and mortality in both developing and developed countries. It is widely believed that there are no undiscovered drug targets in Salmonella enterica, largely due to the high number of nutrients available during infection and redundancy in metabolic pathways. Previous attenuated Salmonella strains on the market attenuate the Salmonella strain metabolically using cya and crp mutations. This mutant cannot compete metabolically with Salmonella and instead vaccinates the animal against that particular Salmonella serovar. However, vaccination is often ineffective especially in the young and the elderly. There are also more than 2600 serovars of Salmonella and vaccination only protects against one. Alternative methods are needed to treat and prevent Salmonella-induced inflammation.

### SUMMARY

Attenuated. Salmonella strains on the market are attenuated metabolically using cya and crp mutations. This mutant cannot compete metabolically with Salmonella and instead vaccinates the animal against that particular Salmonella serovar. However, vaccination is often ineffective especially in the young and the elderly. There are also more than 2600 serovars of Salmonella and vaccination only protects against one. By changing from a vaccination strategy to a probiotic strategy (a strategy in which an avirulent but metabolically competent strain is administered on a regular basis), one can theoretically protect against all 2600 serovars simultaneously, and protect animals or humans in which vaccination is often ineffective (the young and the elderly).

To acquire nutrients in the intestine, Salmonella initiates inflammation, which disrupts the microbiota and causes an oxidative burst that leads to the formation of tetrathionate.

Tetrathionate is used as a terminal electron acceptor for the anaerobic respiration of carbon compounds that otherwise would not be metabolized. One of these carbon sources is ethanolamine, which is derived from host phospholipids. Ethanolamine can be respired by Salmonella, but not fermented. Salmonella actively initiates inflammation using two Type 3 Secretion Systems (T3SS), each encoded within a distinct, horizontally acquired pathogenicity island. SPI1 (Salmonella Pathogenicity Island 1) contributes to invasion of host cells and elicitation of inflammation in the host. SPI2 is required for survival within macrophages and contributes to intestinal inflammation.

As disclosed herein, fructose-asparagine (F-Asn) is a primary nutrient utilized during Salmonella-mediated gastroenteritis. No other organism is known to synthesize or utilize F-Asn. Disclosed are engineered bacteria that can compete with Salmonella for F-Asn and other nutrients and withstand Salmonella-induced inflammation. These bacteria can be used as probiotics to treat and prevent Salmonella-mediated gastroenteritis. An additional advantage is that its use as a probiotic can in some embodiments simultaneously vaccinate the subject against Salmonella.

In some embodiments, the disclosed probiotic comprises an avirulent but metabolically competent. Salmonella bacterium. In some cases, this is accomplished by removing both type 3 secretion systems (T3SS), which are encoded within Salmonella Pathogenicity Islands 1 and 2 (SPI1 and SPI2). These T3SS are required for Salmonella to invade host cells, survive in host cells, to cause inflammation and to cause systemic disease, in some cases, the entire SPI1 and SPI2 loci is deleted. Alternative strategies that would provide the same effect would be to delete individual genes within SRI1 or SPI2. The individual genes within SPI1 and SPI2 encode the structural components of the secretion apparatus, regulatory proteins, and effector proteins that are injected into host cells by the T3SS). Deletion of any single component of the secretion apparatus, regulatory protein, or effector protein may completely disable the function of the T3SS.

Some T3SS effector proteins are encoded outside of SPI1 and SPI2. Deletion of these effector genes may also disrupt the functions of the T3SS. These include sopA, sopB/sigD, sopE, sopE2, srgE, slrP, sopD, sspH1, steA, steB, gogB, pipB, pipB2, sifA, sifB, sopD2, sseI/srfH/gtgB, sseJ, sseK1, sseK2, sseK3, sseL, sspH2, steC, spvB, spvC, spvD, cigR, gtgA, gtgE, pipB2, srfJ, steD, steE. This list continues to grow as more effector genes are discovered.

Another alternative would be to use an organism closely related to Salmonella capable of utilizing many of the same nutrients. This close relative could already be attenuated and/or could be made attenuated, or further attenuated, by deleting virulence genes broadly, or more specifically, genes encoding type 3 secretion systems, if present. Many Salmonella strains that are naturally avirulent, such as Salmonella bongori, could be used in this fashion. Citrobacter, Enterobacter, Cronobacter, and Klebsiella strains may have sufficiently overlapping nutrient sources to be effective, either naturally, or with further attenuation.

The fructose-asparagine utilization system encoded by the fra genes is specific to Salmonella (and possibly some Citrobacter). The fra genes changed the behavior of E. coli Nissle indicating that fructose-asparagine is an important nutrient. However, not all of the changes were good as E. coli Nissle encoding the fra locus gained the ability to kill germ-free C5713116 mice. Adding nutrient acquisition systems, including the fra locus, to other bacteria may enhance the bacterium's ability to compete with Salmonella without the negative effects exhibited by E. coli Nissle. No probiotic species of bacteria are known to utilize fructose-asparagine so all probiotic species could potentially become better able to compete with Salmonella after addition of the fra locus to their genome, as could any of the Citrobacter, Enterobacter, Cronobacter, and Klebsiella.

The Salmonella locus encoding F-Asn utilization, referred to as fra locus, contains the following five genes: fraA (a putative F-Asn transporter), fraB (a putative F-Asn deglycase), fraD (a putative sugar kinase), fraR (a putative transcriptional regulator), and fraE (a putative L-asparaginase). Since these genes encode F-Asn utilization in Salmonella, a recombinant bacterium can be engineered to express genes of the fra locus to confer F-Asn utilization upon a the recombinant bacterium. Therefore, disclosed is a recombinant probiotic bacterium comprising a non-virulent bacterium comprising a heterologous Salmonella gene selected from the group consisting of a fraA gene, fraB gene, fraD gene, fraR gene, fraE gene, or any combination thereof. For example, the heterologous Salmonella gene can be incorporated into a plasmid transfected into the bacterium, or it can be incorporated directly into the chromosome of the bacterium.

In preferred embodiments, the bacterium is a food-grade bacterium that is able to withstand Salmonella-induced inflammation. In some embodiments, the non-virulent Salmonella bacterium lacks phsA, phsB, or phsC, or a combination thereof. The phsABC locus is required for Salmonella to turn a black color on diagnostic agar plates. By deleting these genes, the attenuated Salmonella bacterium used as a probiotic will not cause animals (e.g., chickens) to test positive for Salmonella.

Also disclosed is a composition comprising the recombinant probiotic in a pharmaceutically acceptable or nutraceutically acceptable excipient. In preferred embodiments, the composition contains sufficient colony-forming units (CFU) of the recombinant probiotic to compete with Salmonella for nutrients, such as fructose-asparagine (F-Asn), in the digestive system of the subject. For example, in some embodiments, the composition contains at least 10⁶, 10⁷, 10⁸, or 10⁹ CFU of the recombinant probiotic.

Also disclosed is a method for treating or preventing Salmonella-induced gastroenteritis in a subject, comprising administering to the subject a composition containing the disclosed pharmaceutical or neutraceutical composition.

Also disclosed is a recombinant polynucleotide vector comprising a Salmonella gene selected from the group consisting of a fraA gene, fraB gene, fraD gene, fraR gene, fraE gene, or any combination thereof, incorporated into a heterologous backbone. For example, the polynucleotide vector can be a plasmid. Also disclosed is a bacterium comprising the disclosed recombinant polynucleotide.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plot showing protection of mice against Salmonella serovar Typhimurium strain 14028 by Enterobacter cloacae strain JLD400. Germ-free C5713116 mice were divided into two groups. One group was colonized with 10⁷ cfu of Enterobacter cloacae via the intragastric route (i.g.) and one group was not. One day later both groups were challenged i.g. with 10⁷ cfu of Salmonella. After 24 hours, the cecum and spleen were homogenized and plated to enumerate Salmonella. Each point represents the CFU/g recovered from one mouse with the geometric mean shown by a horizontal line. Statistical significance between select groups was determined by using an unpaired two-tailed Student t test. **=P value <0.01, ***=P value<0.001.

FIG. 2 is a plot showing competitive index (CI) measurements of a sirA mutant in mouse models. Column A shows 10⁷ wild-type MA43 and sirA mutant MA45 in germ-free mice, via the intragastric route (i.g.) and recovered from the cecum after 24 hours. Column B shows 10⁷ wild-type MA43 vs sirA mutant MA45 in germ-free mice mono-associated with Enterobacter cloacae, via the i.g. route and recovered from the cecum after 24 hours. Each point represents the CI from one mouse with the median shown by a horizontal line. Statistical significance of each group being different than 1 was determined by using a one sample Student's t test. Statistical significance between groups was determined using a Mann-Whitney test. *=P value<0.05, ***=P value<0.001.

FIG. 3 is a map of the fra locus of Salmonella enterica. The five genes of the fra locus are shown as grey arrows. The gor and treF genes are shown as black arrows and are conserved throughout the Enterobacteriaceae while the fra locus is not, suggesting that the fra locus was horizontally acquired. The proposed functions and names of each gene are shown below and above the arrows, respectively. The names are based upon the distantly related frl locus of E. coli. For example, the deglycase enzyme of the frl locus is encoded by frlB so the putative deglycase of the fra locus is named fraB. The fra locus has no frlC homolog, while the frl locus does not have an asparaginase. Therefore, the name fraC was not used and the asparaginase was named fraE. The locus tags using the Salmonella nomenclature for strains 14028 (STM14 numbers) and LT2 (STM numbers) are shown above the gene names.

FIG. 4 is a plot showing fitness defect of a fraB1::kan mutant as measured by competitive index (CI) in various genetic backgrounds and mouse models. Column A shows 10⁷ wild-type MA43 and fra B1::kan mutant MA59 in germ-free (GE) C5713116 mice, via the intragastric route (i.g.) and recovered from the cecum after 24 hours. Column B shows 10⁷ wild-type MA43 and fraB1::kan mutant MA59 in germ-free C57BL/6 mice mono-associated with Enterobacter cloacae, via the i.g. route and recovered from the cecum after 24 hours. Column C shows 10⁹ wild-type MA43 and fraB1::kan mutant MA59 in C57BL/6 conventional mice, via the i.g. route and recovered from the cecum after 24 hours. Column D shows 10⁷ wild-type IR715 and fraB1::kan mutant MA59 in streptomycin-treated (ST) C57BL/6 mice, via the i.g. route and recovered from the cecum after 24 hours. Column D shows 10⁷ wild-type IR715 and fraB1::kan mutant MA59 in streptomycin-treated. C57BL/6 mice, via the i.g. route and recovered from the cecum after 4 days. Column F shows Complementation of the fraB1::kan mutation with a plasmid encoding the entire fra island, pASD5006. 10⁷ ASD6090 and ASD6000 in streptomycin-treated C57BL/6 mice, via the i.g. route and recovered from the cecum after 4 days. Column G shows 10⁷ wild-type IR715 and fraB4::kan mutant CS1032 in streptomycin-treated C57BL/6 mice, via the i.g. route and recovered from the cecum after 24 hours. Column H shows 10⁷ wild-type IR715 and fraB1::kan mutant CS1032 in streptomycin-treated C57BL/6 mice, via the i.g. route and recovered from the cecum after 4 days. Column I shows complementation of the fraB4::kan mutation with a plasmid encoding the entire fra island, pASD5006.10⁷ wild-type ASD6090 and fraB4::kan mutant ASD6040 in streptomycin-treated C57BL/6 mice, via the i.g. route and recovered from the cecum after 4 days. Column J shows 10⁷ fra⁺ MA4301 and fraB1::cam mutant MA5900, both strains are a SPI12 SPI22 background, in streptomycin-treated C57BL/6 mice, via the i.g. route and recovered from the cecum after 24 hours. Column K shows 10⁷ fra⁺ MA4301 and fraB1::cam mutant MA5900, both strains are a SPI12 SPI22 background, in streptomycin-treated C57BL/6 mice, via the i.g. route and recovered from the cecum after 4 days. Column L shows 10⁷ fra⁺ MA4301 vs fraB1::cam mutant MA5900, both strains in a SPI12 SPI22 background, in germ-free CS7BL/6 mice, via the i.g. route and recovered from the cecum after 24 hours. Column M shows 10⁷ fra⁺ MA4310 vs fraB1::kan mutant MA5910, both strains are a ttrA2 background, in streptomycin-treated C57BL/6 mice, via the i.g. route and recovered from the cecum after 24 hours. Column N shows 10⁷ fra⁺ MA4310 vs fraB1::kan mutant MA5910, both strains are a ttrA2 background, in streptomycin-treated C57BL/6 mice, via the i.g. route and recovered from the cecum after 4 days. Column 0 shows 10⁷ fra⁺ MA4310 vs fraB1::kan mutant MA5910, both strains are a ttrA2 background, in germ-free C57BL/6 mice, via the i.g. route and recovered from the cecum after 24 hours. Column P shows 10⁴ wild-type MA43 and fraB1::kan mutant MA59 in conventional CS7BL/6 mice, via the intraperitoneal route (i.p.) and recovered from the spleen after 24 hours. Column Q shows 10⁴ wild-type MA43 and fraB1::kan mutant MA59 in streptomycin-treated CS7BL/6 mice, via the i.p. route and recovered from the spleen after 24 hours. Each data point represents the CI from one mouse with the median shown by a horizontal line. Statistical significance of each group being different than 1 was determined by using a one sample Student's t test. Statistical significance between select groups was determined using a Mann-Whitney test. *=P value, 0.05, **=P value, 0.01, ***=P value, 0.001.

FIG. 5 is a bar graph showing histopathology scores of C57BL/6 mice after i.g. inoculation with Salmonella. All groups received 10⁷ cfu except conventional mice (column D), which received 10⁹ cfu. Column A shows germ-free (GF) mice 24 hours post-infection with wild-type MA43 and fraB1::kan mutant MA59; column B shows GF mice 24 hours post-infection with SPI12 SPI22 Salmonella (fra⁺ MA4301 vs fraB1::cam mutant MA5900); column C shows GF mice 24 hours postinfection with ttrA Salmonella (fra+ MA4310 vs fraB1::kan mutant MA5910); column D shows conventional mice 24 hours post-infection with wild-type MA43 and fraB1::kan mutant MA59. Column E shows Strep-treated (ST) mice 4 days post-infection with wild-type IR715 and fraB1::kan mutant MA59; column F ST mice 4 days post-infection with SPI1 SPI2 Salmonella (fra⁺ MA4301 and fraB1::cam mutant MA5900; column G shows ST mice 4 days post-infection with ttrA2 Salmonella (fra⁺ MA4310 vs fraB1::kan mutant MA5910). Error bars represent mean+SD. Statistical significance between select groups was determined using a Mann-Whitney test. *=P value<0.05, **=P value<0.01.

FIG. 6 shows phenotype of a fraB1::kan mutant in the cecum of “humanized” and IL10 knockout mice. 10⁹ wild-type IR715 vs fraB1::kan mutant MA59 in “humanized” Swiss Webster mice (germ free mice inoculated orally with a human fecal sample), or C57BL/6 IL10 knockout mice, as indicated, via the i.g. route and recovered from cecum on day 3 post-infection. FIG. 6A is a plot, where each data point represents the CI from one mouse with the median shown by a horizontal line. Statistical significance of each group being different than 1 was determined by using a one sample Student's t test. ***=P value<0.001. FIG. 6B is a bar graph showing histopathology scores of mice from FIG. 6A. Error bars represent mean+SD.

FIG. 7 is a bar graph showing quantitation of Salmonella in feces on days 1 through 4, and cecum on day 4, post-infection. Groups of five C57BL/6 mice heterozygous for Nramp1 were orally inoculated with 10⁷ CFU of IR715 (wild-type), MA59 (fraB1::kan mutant), or ASD6000 (fraB1::kan mutant with complementation plasmid pASD5006). The geometric mean+SE is shown. Statistical significance between select groups was determined by using an unpaired two-tailed. Student t test. *=P value<0.05, **P=value<0.01.

FIGS. 8A to 8D are graphs showing growth of wild-type and fraB1::kan mutant Salmonella on Amadori products. Growth of wild-type MA43 and fraB1::kan mutant MA59 on F-Asn (FIG. 8A), F-Arg (FIG. 8B), F-Lys (FIG. 8C), asparagine, arginine, lysine, or glucose (FIG. 8D). Bacteria were grown overnight in LB at 37° C. shaking, centrifuged, resuspended in water, and subcultured 1:1000 into NCE medium containing the indicated carbon source at 5 mM. The optical density at 600 nm was then read at time points during growth at 37° C. with shaking. Controls included NCE with no carbon source, and NCE with glucose that was not inoculated, as a sterility control (FIG. 8D). FIG. 8E is a graph showing complementation of a fraB1::kan mutation with plasmid pASD5006 encoding the fra island (ASD6000) or the vector control, pWSK29 (ASD6010). Each point in FIGS. 8A-8E represents the mean of three cultures with error bars indicating standard deviation. FIG. 8F shows the structure of F-Asn (CAS#34393-27-6).

FIG. 9 is a graph showing growth of Salmonella on F-Asn as sole nitrogen source. Growth of wild-type MA43 and fraB1::kan mutant MA59 on F-Asn. Bacteria were grown overnight in LB at 37° C. shaking, centrifuged, resuspended in water, and subcultured 1:1000 into NCE medium lacking a nitrogen source (NCE-N) but containing the indicated carbon source at 5 mM. The optical density at 600 nm was then read at time points during growth at 37° C. with shaking. Controls included NCE-N with no carbon source, NCE-N with 5 mM glucose, and NCE-N with glucose that was not inoculated, as a sterility control. Each point represents the mean of four cultures and error bars represent standard deviation.

FIGS. 10A to 10D show growth of Salmonella on F-Asn in the presence or absence of tetrathionate or oxygen. Growth of wild-type MA43 and fraB1::kan mutant MA59 on 5 mM F-Asn or 5 mM glucose anaerobically (FIGS. 10A and 10B) or aerobically (FIGS. 10C and 10D) in the presence (FIGS. 10A and 10C) or absence (FIGS. 10B and 10D) of 40 mM tetrathionate (S406 22). Bacteria were grown overnight in LB at 37° C. shaking, centrifuged, resuspended in water, and subcultured 1:1000 into NCE medium containing the indicated carbon source. The optical density at 600 nm was then read at time points during growth at 37° C. with shaking. Each point represents the mean of four cultures with error bars indicating standard deviation.

FIG. 11 is a plot showing competitive index (CI) measurements of a fraB1::kan mutant during in vitro growth. Cultures were grown overnight in LB, pelleted and washed in water, subcultured 1:10,000 and grown for 24 hours at 37° C. in NCE minimal medium containing 5 mM F-Asn, aerobically or anaerobically, in the presence or absence of tetrathionate (S₄O₆²⁻), as indicated. Column A shows anaerobic growth in the presence of tetrathionate; column B shows anaerobic growth in the absence of tetrathionate; column c shows aerobic growth in the presence of tetrathionate; column D shows aerobic growth in the absence of tetrathionate. Each data point represents the CI from one culture with the median shown by a horizontal line. Statistical significance of each group being different than 1 was determined by using a one sample Student's t test. Statistical significance between select groups was determined using a Mann-Whitney test. **=P value<0.01, ***=P value<0.001.

FIG. 12 is a proposed model of Fra protein localization and functions. A proteomic survey of subcellular fractions of Salmonella previously identified FraB (the deglycase) as cytoplasmic and FraE (the asparaginase) as periplasmic. Therefore, it is possible that F-Asn is converted to F-Asp in the periplasm by the asparaginase and that the transporter and kinase actually use F-Asp as substrate rather than F-Asn. The FraD kinase of Salmonella shares 30% amino acid identity with the FrlD kinase of E. coli. FrlD phosphorylates F-Lys to form F-Lys-6-P. Therefore, FraD may phosphorylate F-Asp to form F-Asp-6-P. The FrlB deglycase of E. coli shares 28% amino acid identity with FraB of Salmonella. The FrlB deglycase converts F-Lys-6-P to lysine and glucose-6-P [Wiame E, et al. (2002) J Biol Chem 277:42523-42529], FraB of Salmonella may convert F-Asp-6-P to aspartate and glucose-6-P.

FIG. 13 is a graph showing growth (OD600) of Nissle+vector (ASD9000) or Nissle+ fra (ASD9010) in M9 minimal medium containing either 5 mM glucose or 5 mM F-Asn as carbon source.

FIGS. 14A and 14B are graphs showing evaluation of probiotics as prophylactics in germ-free mice. On consecutive days groups of five germ-free C57BL/6 mice (FIG. 14A) or germ-free Swiss Webster mice (FIG. 14B) were orally administered a probiotic strain (10⁹ CFU) or sham (water), and then virulent Salmonella (10⁴ CFU of JLD1214). Survival was monitored over time. Statistical significance of each treatment compared to sham was determined with log-rank (Mantel-Cox) tests with a P value<0.05 considered significant. In FIGS. 14A and 14B, the sham was statistically different than all of the treatments.

FIGS. 15A and 15B are graphs showing safety of probiotics in germ-free C57BL/6 mice (FIG. 15A) and germ-free Swiss Webster mice (FIG. 15B). Groups of five mice were orally administered a probiotic strain (10⁹ CFU) and survival was monitored over time.

FIG. 16 is a graph showing evaluation of probiotics as prophylactics in strep-treated Swiss Webster mice. On consecutive days groups of 15 mice were administered streptomycin, then a probiotic strain (10⁹ CFU) or sham (water), and then virulent Salmonella (10⁷ CFU of JLD1214). Survival was monitored over time. Statistical significance of each treatment compared to sham was determined with log-rank (Mantel-Cox) tests with a P value<0.05 considered significant. The sham is statistically different than all treatments except Nissle+ vector.

FIG. 17 is a plot showing CBA/J mice orally inoculated with 10⁹ CFU of virulent Salmonella strain JLD1214. Ten days post-infection, groups of five mice were treated with 10⁹ CFU of probiotic or sham, Salmonella (JLD1214) shedding in feces was measured on days 10 (just before probiotic inoculation), 11, and 13. Salmonella (JLD1214) in the ceca was measured on day 17.

FIG. 18 is a plot showing CBA/J mice orally inoculated with 10⁹ CFU of virulent Salmonella strain JLD1214. Groups of eight mice were treated with 10⁹ CFU of probiotic or sham three times, on days 10, 12, and 14 post-infection. Salmonella (JLD1214) shedding in feces was measured on the same days just before probiotic inoculation. Salmonella (JLD1214) in the ceca was measured on day 15.

FIGS. 19A and 19B are bar graphs showing mRNA expression level of inflammatory marker genes IFNγ (FIG. 19A) and TNFα (FIG. 19B), as measured by qRT-PCR, from ceca harvested from the mice in FIG. 6 on day 15 post-infection. The error bars are mean+/−SEM.

FIG. 20 is a plot of histopathology scores from ceca harvested from the mice in FIGS. 20A and 20B on day 15 post-infection. The bar represents the median.

### DETAILED DESCRIPTION

Disclosed are engineered bacteria that can compete with Salmonella for F-Asn and other nutrients and withstand Salmonella-induced inflammation.

Avirulent but Metabolically Competent Salmonella Bacterium

In some embodiments, the probiotic comprises an avirulent but metabolically competent Salmonella bacterium. In some cases, this is accomplished by removing both type 3 secretion systems (T3SS), which are encoded within Salmonella Pathogenicity Islands 1 and 2 (SPI1 and SPI2). These T3SS are required for Salmonella to invade host cells, survive in host cells, to cause inflammation and to cause systemic disease. In some cases, the entire SPI1 and SPI2 loci is deleted. Alternative strategies that would provide the same effect would be to delete individual genes within SPI1 or SPI2. The individual genes within SPI1 and SPI2 encode the structural components of the secretion apparatus, translocases, or chaperones (sicA, sicP, invA, invB, invC, invI, invJ, invE, invG, invH, orgA, orgB, orgC, prgH, prgK, prgI, prgJ, spaM, spaN, spaO, spaP, spaQ, spaR, spaS, sipA, sipD, sipB, sipC, sseC, sseD, sseB, ssaU, ssaT, ssaS, ssaR, ssaQ, ssaP, ssaO, ssaG, ssaJ, ssaC, ssaV, ssaN, spiC, ssaL, ssaM, ssaK, ssaI, ssaH, ssaG, ssaB, ssaC, ssaD, ssaE, sscA, sscB, sseA, sseE, sseG, sseF), regulatory proteins (hilA, invF, hilC, hilD, iagB, sprA, sprB, ssrA, ssrB), and effector proteins (proteins that are injected into host cells by the T3SS, sptP, avrA, sicP, iacP). Deletion of any single component of the secretion apparatus, translocase, chaperone, regulatory protein, or effector protein may completely disable the function of the T3SS.

Some T3SS effector proteins are encoded outside of SPI1 and SPI2. Deletion of these effector genes may also disrupt the functions of the T3SS. These include sopA, sopB/sigD, sopE, sopE2, srgE, slrP, sopD, sspH1, steA, steB, gogB, pipB, pipB2, sifA, sifB, sopD2, sseI/srfH/gtgB, sseJ, sseK1, sseK2, sseK3, sseL, sspH2, steC, spvB, spvC, spvD, cigR, gtgA, gtgE, pipB2, srfJ, steD, steE. This list continues to grow as more effector genes are discovered.

Another alternative would be to use an organism closely related to Salmonella capable of utilizing many of the same nutrients. This close relative could already be attenuated and/or could be made attenuated, or further attenuated, by deleting virulence genes broadly, or more specifically, genes encoding type 3 secretion systems, if present. Many Salmonella strains that are naturally avirulent, such as Salmonella bongori, could be used in this fashion. Citrobacter, Enterobacter, Cronobacter, and Klebsiella strains may have sufficiently overlapping nutrient sources to be effective, either naturally, or with further attenuation. In some embodiments, combinations of 2, 3, 4, 5, 6 or more strains of Salmonella, Citrobacter, Enterobacter, Cronobacter, and/or Klebsiella are used.

The disclosed probiotic can also contain one or more intestinal microorganism, such as those commonly found in digestive health probiotic supplements. For example, in some embodiments, the probiotic further contains one or more microorganisms selected from the group consisting of Lactobacillus acidophilus, Lactobacillus bulgaricus, Lactobacillus casei, Lactobacillus fermentum, Lactobacillus gasseri, Lactobacillus plantarum, Lactobacillus reuteri, Lactobacillus rhamnosus (e.g., GG), Lactobacillus paracasei, Lactobacillus plantarus (299v), Lactobacillus rhamnosus, Lactobacillus reuteri, Lactobacillus salivarius (e.g., UCC4331), Bifidobacterium animalis (DN-173010), Bifidobacterium breve, Bifidobacterium bifidum, Bifidobacterium longum, Bifidobacterium infantis, Bacillus coagulans, Saccharomyces boulardii, Streptococcus thermophiles, Streptoccocus salivarius K12, and Streptoccocus Salivarius M18.

Bacterium with Heterologous Fra Locus

In some embodiments, the probiotic comprises a recombinant probiotic bacterium comprising a non-virulent bacterium comprising a heterologous Salmonella gene selected from the group consisting of a fraA gene, fraB gene, fraD gene, fraR gene, fraE gene, or any combination thereof. The Salmonella locus encoding F-Asn utilization, referred to as fra locus, contains the following five genes: fraA (a putative F-Asn transporter), fraB (a putative F-Asn deglycase), fraD (a putative sugar kinase), fraE (a putative L-asparaginase), and fraR (a putative transcriptional regulator). Since these genes encode F-Asn utilization in Salmonella, a recombinant bacterium can be engineered to express genes of the fra locus to confer F-Asn utilization on the recombinant bacterium. Also disclosed is a recombinant polynucleotide vector comprising a Salmonella gene selected from the group consisting of a fraA gene, fraB gene, fraD gene, fraE gene, fraR gene, or any combination thereof, incorporated into a heterologous backbone.

In some embodiments, the fraA gene has the following nucleic acid sequence:

(SEQ ID NO: 1)
atgttttggacggaattatgttttatccttgtggccctgatgataggc
gccaggatcggcggcgtatttttagggatggtcggcgggttaggcgtc
ggcgtgatggtttttatttttggcctgacgccttctacgccaccgatt
gatgttattctgattattctttctgttgtcctggcggccgcttcttta
caggcctccggcgggctggatttactggtcaaactggcggaaaaaatt
ctgcgtcgccacccgcgttacattacgttattagcgccgtttatctgt
tatatcttcacttttatgtcaggaacggggcatgtcgtttatagcttg
ctaccggttatttctgaagtcgcacgggattcaggtattcgaccggaa
cgtcctttatctatttccgttatcgcatcgcaacaggcgatcaccgcc
agtcctatatctgccgccatggcggcgatgattggtttaatggcgccg
ttgggcgtctctatttcaaccattatgatgatttgcgtgcccgccacg
ttaatcggcgtagcgatgggggcaatagcgacctttaataaaggaaaa
gagttaaaagacgatccggaatatcaacgtcggcttgctgaagggtta
attaaacctgcgcagaaagaaagtaaaaatacggtggtcacttcgcgc
gccaaattgtcggtggcgttatttctgaccagtgcgatcgttatcgtt
ctgttaggactgattccggcgctgcggcccatggtggaaacagcgaaa
gggctacaaccgctttcgatgtccgccgctatccagattacgatgctc
tcttttgcctgcctgattgtgttgttatgccgaccgcaggtcgatcaa
attatcagcggtacggtatttcgggcgggcgcgctggcgattgtctgc
gccttcggcctggcctggatgagtgagacgttcgtgaatggtcatatc
gcgttgattaaggcagaagtgcaaactctattgcaacagcatacctgg
cttatcgccattatgatgttttttgtgtccgctatggtcagcagccag
gcggcaacgacggttaattctgttgccgctggggctggcgttagggtt
gcccgcttatgcattaatcggctcctggcctgccgttaacggctattt
ctttattccggtggcggggcagtgtctggcggcgctggcgtttgacga
taccggtacgacgcgtattggcaaatatgtgcttaaccatagttttat
gcgtccgggattagttaacgtgattgtctcggtcattgtcgggctgtt
aataggaaaaatggttctggcctga.

In some embodiments, the fraA gene has a nucleic acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:1.

In some embodiments, the fraA gene encodes the following amino acid sequence:

(SEQ ID NO: 2)
MFWTELCFILVALMIGARIGGVFLGMVGGLGVGVMVFIFGLTPSTPPI
DVILIILSVVLAAASLQASGGLDLLVKLAEKILRRHPRYITLLAPFIC
YIFTFMSGTGHVVYSLLPVISEVARDSGIRPERPLSISVIASQQAITA
SPISAAMAAMIGLMAPLGVSISTIMMICVPATLIGVAMGAIATFNKGK
ELKDDPEYQRRLAEGLIKPAQKESKNTVVTSRAKLSVALFLTSAIVIV
LLGLIPALRPMVETAKGLQPLSMSAAIQITMLSFACLIVLLCRPQVDQ
IISGTVFRAGALAIVCAFGLAWMSETFVNGHIALIKAEVQTLLQQHTW
LIAIMMFFVSAMVSSQAATTLILLPLGLALGLPAYALIGSWPAVNGYF
FIPVAGQCLAALAFDDTGTTRIGKYVLNHSFMRPGLVNVIVSVIVGLL
IGKMVLA.

In some embodiments, the fraA gene encodes an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%. 92%, 93%, 94%, 95%, 96%, 97%, 98%, or 99% sequence identity with SEQ ID NO:2.

In some embodiments, the fraB gene has the following nucleic acid sequence:

(SEQ ID NO: 3)
atgatgggtatgaaagagacagttagcaatattgtgaccagccaggca
gagaaaggaggcgttaaacacgtctattacgtggcgtgcggcggttct
tatgcggcgttctatccggcgaaagcatttttagaaaaagaagcgaaa
gcgttgactgtcggtctgtataacagcggagaatttattaacaacccg
ccggtagcgctgggagaaaatgccgttgtggttgtcgcctcccacaaa
ggtaatacgccagagacaattaaagcggctgaaatcgcccgtcagcac
ggcgcgccggtcattggtttaacctggataatggattcaccgttggtg
gcgcattgcgactatgtggaaacgtacacgtttggcgacggtaaagat
attgccggagagaaaacgatgaaaggcctgctgagtgcggtcgaactg
ctccagcagacggaagggtatgcgcactacgacgattttcaggatggc
gtcagcaaaatcaaccgtatcgtctggcgcgcttgcgagcaggtagcg
gagcgtgcgcaggcgttcgcgcaggaatataaagacgataaagtcatt
tataccgtcgccagcggcgcgggctatggcgcagcctacctacagagc
atctgcatctttatggaaatgcaatggatacattccgcctgtattcat
agcggtgagtttttccacgggccgtttgaaattaccgatgcgaatacg
cctttcttcttccagttttccgagggcaatacgcgggcggtggatgaa
cgcgcgttaaacttcctgaaaaaatatggccgccggattgaagttgtc
gatgcgaaagaactggggctatcgaccattaaaaccacggttattgat
tactttaaccactctctctttaataacgtttatcccgtttacaatcgg
gcgttagctgaggcgcgtcagcatccgttaacgacgcgccgctatatg
tggaaagtggaatattaa.

In some embodiments, the fraB gene has a nucleic acid sequence having at east 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:3.

In some embodiments, the fraB gene encodes the following amino acid sequence:

(SEQ ID NO: 4)
MMGMKETVSNIVTSQAEKGGVKHVYYVACGGSYAAFYPAKAFLEKEAK
ALTVGLYNSGEFINNPPVALGENAVVVVASHKGNTPETIKAAEIARQH
GAPVIGLTWIMDSPLVAHCDYVETYTFGDGKDIAGEKTMKGLLSAVEL
LQQTEGYAHYDDFQDGVSKINRIVWRACEQVAERAQAFAQEYKDDKVI
YTVASGAGYGAAYLQSICIFMEMQWIHSACIHSGEFFHGPFEITDANT
PFFFQFSEGNTRAVDERALNFLKKYGRRIEVVDAKELGLSTIKTTVID
YFNHSLFNNVYPVYNRALAEARQHPLTTRRYMWKVEY.

In some embodiments, the fraB encodes an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:4.

In some embodiments, the fraD gene has the following nucleic acid sequence:

(SEQ ID NO: 5)
atgagcatcagcgtattgggtattggcgacaacgttgtcgataaatac
ctgcattccggcatcatgtaccccggcggtaatgcattaaattttgct
gtctatgcgaaattagcagacatccccagcgcgtttatgggggcgttt
ggcaatgacgacgccgcgcagcacgtacaggatgtattacaccagcta
cagatagacatctctcacagccgccattataccggcgaaaatgggtat
gcctgtatccgtctctcgcatggcgatcggcaatttgtcgccagcaac
aaaaacggcgtattgcgggaacatccttttagtctgtctgacgacgat
cttcgctatatatcacaatttaccttagtccattccagtattaacggc
cacctggaatcggaactggagaaaattaaacaacaaaccgtcttactc
tcttttgatttttccgggcgcggtacagacgactattttgaaaaggta
tgcccgtgggtagattacggatttatctcctgtagcgggttatcgcca
gatgaaatcaaagtaaaactcaataaactttatcgttatggctgtcgg
catattattgccacctgcgggcatgaaaaagtttattatttttccggc
gcggattatctggagtggcaacctgcttatatcgaacctgtcgatacg
ctgggcgcaggcgacgccttcttaaccggttttttgctttccattttg
caatcgggtatggcggaacccgataaagaaagcgtgttacgcgccatg
cggcagggcgggaaatcggcggcgcaggtgttatctcattacggcgca
tttggttttggtaaaccgtttgcacaatag.

In some embodiments, the fraD gene has a nucleic acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ NO:5.

In some embodiments, the fraD gene encodes the following amino acid sequence:

(SEQ ID NO: 6)
MSISVLGIGDNVVDKYLHSGIMYPGGNALNFAVYAKLADIPSAFMGAF
GNDDAAQHVQDVLHQLQIDISHSRHYTGENGYACIRLSHGDRQFVASN
KNGVLREHPFSLSDDDLRYISQFTLVHSSINGHLESELEKIKQQTVLL
SFDFSGRGTDDYFEKVCPWVDYGFISCSGLSPDEIKVKLNKLYRYGCR
HIIATCGHEKVYYFSGADYLEWQPAYIEPVDTLGAGDAFLTGFLLSIL
QSGMAEPDKESVLRAMRQGGKSAAQVLSHYGAFGFGKPFAQ.

In some embodiments, the fraD encodes an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:6.

In some embodiments, the fraE gene has the following nucleic acid sequence:

(SEQ ID NO: 7)
atgaaaattagagttttcatggccaccgtgttgctgctcatcagccac
tgtgtatttagcacaacgtcactaccgcatattgttattctcgcgaca
ggtggtactatcgccgggacggcagccaataatacgcaaaccgccgga
tataaatctggtgaacttggcgtgcaaacattaataaatgaagtgccg
gaaatgaataatatcgctcgcgttgacggcgagcaggtggcgaatatt
ggtagcgaaaatatgaccagcgatatcatcctgaaactttcacagaag
gtgaatgcgttattggcgcgggacgatgttgacggtgtggttattact
catggcactgacacgctcgatgaaaccgcctactttcttaatttgacc
gtgaaaagcgacaaaccggtggtgtttaccgctgcaatgcggcccgcg
tcggcaatcagcgccgatggcgcaatgaacctgctggaagcggtcacg
gtggctgctgacccgaatgcgaagggacgcggtgtgatggtggtttta
aacgatcgtattggttcggcgcgctttgtgacgaaaactaatgccacg
actctggatacctttaaagcgccggaagagggctatctgggggtcatc
gttaatggtcagccacagttcgaaacgcgggtggaaaaaattcatacc
ctgcgatctgtttttgacgtacgtaatatcaaaaaattacccaatgtg
gtgattatttacggctatcaggacgacccggaatatatgtatgatgcg
gcgatcgcccatcacgcggacggtattatttatgccggaaccggcgca
ggttcggtctcggtacgcagcgacgcggggattaaaaaagcggagaaa
gccgggattatcgtggtgcgcgcttcccgcaccggaaacggcgtcgta
ccgttggataaagggcagccagggctggtgtctgactcgctcaacccg
gcgaaggcgcgagtcttgctgatgacggcattaactcagacgcgtaat
ccggaactgatccagagttatttcagtacgtattaa.

In some embodiments, the fraE gene has a nucleic acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:7.

In some embodiments, the fraE gene encodes the following amino acid sequence:

(SEQ ID NO: 8)
MKIRVFMATVLLLISHCVFSTTSLPHIVILATGGTIAGTAANNTQTAG
YKSGELGVQTLINAVPEMNNIARVDGEQVANIGSENMTSDIILKLSQK
VNALLARDDVDGVVITHGTDTLDETAYFLNLTVKSDKPVVFTAAMRPA
SAISADGAMNLLEAVTVAADPNAKGRGVMVVLNDRIGSARFVTKTNAT
TLDTFKAPEEGYLGVIVNGQPQFETRVEKIHTLRSVFDVRNIKKLPNV
VIIYGYQDDPEYMYDAAIAHHADGIIYAGTGAGSVSVRSDAGIKKAEK
AGIIVVRASRTGNGVVPLDKGQPGLVSDSLNPAKARVLLMTALTQTRN
PELIQSYFSTY.

In some embodiments, the fraE encodes an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:8.

In some embodiments, the fraR gene has the following nucleic acid sequence:

(SEQ ID NO: 9)
atgatcgagcaacccgacagtaaaagcgccaaaccgctttataagcag
cttgaagccgccttaaaagaggctattgcgcgtggagagtataaacca
ggccagcagatcccgacggaaaatgaactgagcgtgcgctggcaggtg
agcagggtcacggtccgtaaggcgctggatgcgctgacgcgtgaaaat
ttgctgacccgtgtctccggcaaaggcacctttgtctctggtgagaaa
tttcagcgcagcatgaccggcatcatgagtttcagcgagttatgccag
tcccagggacgtcgcccggggtcacgcaccatcaaatccgtttttgaa
tcggtagacgatgagacaaaagcgttactgaatatgaacgatggcgaa
aaagcggtcgtcattgaacgtatccgctatgccgacgatgtggcggta
tcgctggaaaccgtacatcttcccccacgttttgcgtttttgctggac
gaagatcttaataatcactctttgtatgaatgcttacgcgagaaatac
catttatggtttacccactcccgtaagatgatcgaactggtttatgcc
agctttgaagtcgcccattatcttggcgtcaacgagggttatccgctg
atcctgataaaaagtgaaatgattgataacaaaggagaactctcctgc
gtttcgcaacagttgattgtcggcgataaaatacggtttaccgtatga.

In some embodiments, the fraR gene has a nucleic acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:9.

In some embodiments, the fraR gene encodes the following amino acid sequence:

(SEQ ID NO: 10)
MIEQPDSKSAKPLYKQLEAALKEAIARGEYKPGQQIPTENELSVRWQV
SRVTVRKALDALTRENLLTRVSGKGTFVSGEKFQRSMTGIMSFSELCQ
SQGRRPGSRTIKSVFESVDDETKALLNMNDGEKAVVIERIRYADDVAV
SLETVHLPPRFAFLLDEDLNNHSLYECLREKYHLWFTHSRKMIELVYA
SFEVAHYLGVNEGYPLILIKSEMIDNKGELSCVSQQLIVGDKIRFTV.

In some embodiments, the fraR encodes an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO:10.

Any food-grade bacterium that is able to withstand Salmonella-induced inflammation and that expresses, or can be engineered to express,fra locus genes and/or compete with Salmonella fructose-asparagine (F-Asn) nutrients can be used in the disclosed compositions and methods. The fra genes changed the behavior of E. coli Nissle indicating that fructose-asparagine is an important nutrient. However, not all of the changes were good as E. coli Nissle encoding the fra locus gained the ability to kill germ-free C57BL/6 mice. Adding nutrient acquisition systems, including the fra locus, to other bacteria may enhance the bacterium's ability to compete with Salmonella without the negative effects exhibited by E. coli Nissle. No probiotic species of bacteria are known to utilize fructose-asparagine so all probiotic species could potentially become better able to compete with Salmonella after addition of the fra locus to their genome, as could any of the Citrobacter, Enterobacter, Cronobacter, and Klebsiella.

Therefore, in some cases, the probiotic species engineered to express the fra locus is an intestinal microorganism, such as those commonly found in digestive health probiotic supplements. For example, in some embodiments, the engineered probiotic is a microorganisms selected from the group consisting of Lactobacillus acidophilus, Lactobacillus bulgaricus, Lactobacillus casei, Lactobacillus fermentum, Lactobacillus gasseri, Lactobacillus plantarum, Lactobacillus reuteri, Lactobacillus rhamnosus (e.g., GG), Lactobacillus paracasei, Lactobacillus plantarus (299v), Lactobacillus rhamnosus, Lactobacillus reuteri, Lactobacillus salivarius (e.g., UCC4331), Bifidobacterium animalis (DN-173010), Bilidobacterium breve, Bifidobacterium bifidum, Bifidobacterium longum, Bifidobacterium infantis, Bacillus coagulans, Saccharomyces boulardii, Streptococcus thermophiles, Streptoccocus salivarius K12, and Streptoccocus Salivarius M18.

Gene Expression Systems

Suitable vectors for expressing heterologous genes in bacteria can be chosen or constructed containing appropriate regulatory sequences, including promoter sequences, terminator fragments, enhancer sequences, marker genes and other sequences as appropriate. Vectors may be plasmids, viral, e.g., phage or pliagemid, as appropriate. For further details see, for example, Molecular Cloning, a Laboratory Manual: 2nd edition, Sambrook et al., 1989, Cold Spring Harbor Laboratory Press. Many known techniques and protocols for manipulation of nucleic acid, for example, in preparation of nucleic acid constructs, mutagenesis, sequencing, introduction of DNA into cells and gene expression, and analysis of proteins, are described in detail in Short Protocols in Molecular Biology, Second Edition, Ausubel et at eds., John Wiley & Sons, 1992. The disclosures of Sambrook et al. and Ausubel et al. are incorporated herein by refrence.

The coding sequences forfra locus genes) may be contained in an operon, i.e., a nucleic acid construct for multi-cistronic expression. In an operon, transcription from the promoter results in an mRNA which comprises more than one coding sequence, each with its own suitably positioned ribosome binding site upstream. Thus, more than one polypeptide can be translated from a single mRNA. Use of an operon therefore enables expression of more than one biologically active polypeptide by the bacterium of the present invention. Alternatively, the coding sequences for two separate biologically active polypeptides can be part of the same nucleic acid vector, or separate vectors, where they are individually under the regulatory control of separate promoters. The promoters may be the same or different.

The promoter can be expressed constitutively in the bacterium. Use of a constitutive promoter avoids the need to supply an inducer or other regulatory signal for expression to take place. The promoter may be homologous to the bacterium employed, i.e., one found in that bacterium in nature. For example, a promoter that is functional in E. coli may be used. The promoter could be, by way of a non-limiting example, the bla or cat promoters, or the lambda right phage promoter, which are all functional in E. coli.

Probiotic Composition

Also disclosed are pharmaceutical and/or nutraceutical formulations. Such formulations comprise a prophylactically or therapeutically effective amount of a disclosed bacterium and a pharmaceutically or nutraceutically acceptable carrier.

In some cases, the composition comprises a carrier to facilitate the probiotics being delivered to the gastro-intestinal tract (e.g., the small intestine) in a viable and metabolically-active condition. In some embodiments, the bacterium are also delivered in a condition capable of colonizing and/or metabolizing and/or proliferating in the gastrointestinal tract.

In some embodiments, the composition is a foodstuff. In this regard, the term “foodstuff as used herein includes liquids (e.g., drinks), semi-solids (e.g., gels, jellies, yoghurt, etc) and solids. Exemplary foodstuffs include dairy products, such as fermented milk products, unfermented mild products, yoghurt, frozen yoghurt, cheese, fermented cream, milk-based desserts milk powder, milk concentrate or cheese spread. Other products are also contemplated, such as soy-based products, oat-based products, infant formula, and toddler formula. The composition can also be presented in the form of a capsule, tablet, syrup, etc. For example, the composition can be a pharmaceutical composition. Such a composition can comprise a pharmaceutically acceptable carrier, e.g., to facilitate the storage, administration, and/or the biological activity of the probiotic (see, e.g., Remington's Pharmaceutical Sciences, 16th Ed., Mac Publishing Company (1980). Suitable carriers for the present disclosure include those conventionally used, e.g., water, saline, aqueous dextrose, lactose, a buffered solution, starch, cellulose, glucose, lactose, sucrose, gelatin, malt, rice, flour, and the like. In some embodiments, the carrier provides a buffering activity to maintain the probiotic at a suitable pH to thereby exert a biological activity.

In a liquid therapeutic composition, the food-grade bacterium can be in suspension in a liquid that ensures physiological conditions for a probiotic bacterium. In a solid therapeutic composition, the food-grade bacterium can be present in free, preferably lyophilized form, or in immobilized form. For example, the food-grade bacterium can be enclosed in a gel matrix which provides protection for the cells.

In preferred embodiments, the composition contains sufficient colony-forming units (CFU) of the recombinant probiotic to compete with Salmonella for fructose-asparagine (F-Asn) as a nutrient in the digestive system of the subject. For example, in some embodiments, the composition contains at least 10⁶, 10⁷, 10⁸, or 10⁹ CFU of the recombinant probiotic, including about 10⁹ CFU of the recombinant probiotic.

Generally, the disclosed ingredients of formulations are supplied either separately or mixed together in unit dosage form, for example, as a dry lyophilized powder or water free concentrate in a hermetically sealed container such as an ampoule or sachet indicating the quantity of active agent.

A solid therapeutic composition intended for oral administration is preferably provided with a coating resistant to gastric juice. it is thereby ensured that the food-grade bacterium contained in the therapeutic composition can pass through the stomach unhindered and undamaged and the release of the food-grade bacterium first takes place in the upper intestinal regions. For example, the disclosed bacterium can be encapsulated, e.g., microencapsulated Encapsulation of the probiotic can enhance survival in the gastric and/or gastrointestinal tract of a subject. Reagents and methods of encapsulation are known in the art andlor described herein. Exemplary reagents for encapsulation include alginate. Alginate is one of the most commonly used reagents for encapsulation of probiotics. Alginate is a linear polysaccharide consisting of 1→4 linked P-(D)-glucuronic (G) and a-(L)-mannuronic (M) acids generally derived from brown algae or bacterial sources. It is commercially available in a wide range of molecular weights from tens to hundreds of kilodaitons and is well suited to bacterial encapsulation due to its mild gelling conditions, GRAS (generally recognized as safe) status, and substantial lack of toxicity.

Alginate gels upon contact with divalent metals (e.g. calcium, cadmium or zinc). This ability has been exploited to form microcapsules using an extrusion process. This process involves the dropping of a concentrated alginate solution, most commonly through a needle, into calcium chloride solution, externally gelling the polymer into a microcapsule. The size of the microcapsules formed using external gelation is governed by the size of droplets formed during the extrusion process, with particles from as little as tens of microns being produced by spray technology, up to millimetre size when needle extrusion is used.

Another approach which is commonly used is the emulsion method. In this process microcapsules are formed by the formation of a water-in-oil emulsion, usually stabilized by surfactants, such as Tween 80, with the alginate being dissolved in the water phase. The alginate is usually then gelled by external gelation, i.e. the addition of calcium chloride solution to the emulsion. Alternatively, microcapsules may be formed by internal gelation, in which the alginate in solution contains calcium carbonate. An organic acid is added to this emulsion, and as it penetrates into the discrete water phase it reacts with the calcium carbonate forming calcium ions and carbonic acid, resulting in the gelation of the alginate.

The coating, or incorporation, of other materials into the alginate microcapsules can also be useful for probiotic microencapsulation research. Along with the protection that such coatings can offer to the microorganisms, other beneficial properties may also be imparted, such as giving greater control over release. A common coating material is the polysaccharide chitosan. Chitosan is a natural, linear cationic polysaccharide containing both glucosamine and N-acetyl glucosamine residues. Chitosan is the (usually partially) N-deacetylated form of chitin, a natural mucopolysaccharide derived from some natural supporting structures, such as the exoskeletons of crustaceans.

Other casting materials that can be combined with the alginate (or other encapsulating reagent) include whey protein, palm oil, xanthan gum, cellulose acetate phthalate or, starch.

Other polysaccharides that have been used to encapsulate probiotics include xanthan gum, gum acacia, guar gum, locust bean gum, and carrageenan.

Method

Also disclosed is a method for treating or preventing Salmenella-induced gastroenteritis in a subject, comprising administering to the subject a composition containing a disclosed pharmaceutical or neutraceutical priobiotic composition.

Salmonella species are facultative intracellular pathogens. Many infections are due to ingestion of contaminated food. They can be divided into two groups—typhoidal and nontyphoidal Salmonella serovars. Nontyphoidal serovars are more common, and usually cause self-limiting gastrointestinal disease. They can infect a range of animals, and are zoonotic, meaning they can be transferred between humans and other animals. Typhoidal serovars include Salmonella Typhi and Salmonella Paratyphi A, which are adapted to humans and do not occur in other animals.

Infection with nontyphoidal serovars of Salmonella will generally result in food poisoning. The organism enters through the digestive tract and must be ingested in large numbers to cause disease in healthy adults. An infectious process can only begin after living salmonellae (not only their toxins) reach the gastrointestinal tract. Some of the microorganisms are killed in the stomach, while the surviving salmonellae enter the small intestine and multiply in tissues (localized form). Salmonella injects effector proteins into host cells to elicit an inflammatory response.

The disclosed probiotic may be administered on a daily basis or more or less often, depending on the survival of the probiotic in the subject. In some embodiments, the probiotic is administered with food or within three hours or two hours or one hour of consuming food. Consuming the probiotic with food or soon thereafter is likely to increase the survival of the probiotic by increasing the pH of the acidic components of the gastric or gastrointestinal tract. In one example, the probiotic is administered in an effective amount or a therapeutically effective amount or a prophylactically effective amount.

In one example, the method comprises administering the probiotic, encapsulated form thereof or composition in an effective amount of at least about 10⁴ to about 10¹⁰ CFU per dose; or about 10⁵ to about 10⁹ CFU per dose; or about 10⁵ to about 10⁷ CFU per dose; or about 10⁹ CFU per dose.

Definitions

The term “encapsulate” refers to the coating of a probiotic or a plurality of probiotics in a composition. In one example, the probiotic is encapsulated in a composition that protects the probiotic from gastric conditions and, for example, that releases the probiotic in the intestine, such as the small intestine, of a subject.

The term “lactic acid bacterium” designates a bacterium of the group of Gram-positive, catalase negative, non-motile, microaerophilic or anaerobic bacteria which ferment sugar with the production of acids including lactic acid as the predominantly produced acid, acetic acid, formic acid and propionic acid. Exemplary lactic acid bacteria are found among Lactococcus species (including Lactococcus lactis), Streptococcus species, Enterococcus species, Lactobacillus species, Leuconostoc species, Oenococcus species, and Pediococcus species.

The term “nutraceutically acceptable” refers to those compounds, materials, compositions, and/or dosage forms which are compatible with the other ingredients of the formulation and suitable for ingestion by mammals, such as humans.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “probiotic bacterium” denotes a natural or recombinant bacterium which ingested live in adequate quantities can exert beneficial effects on the human health. They are now widely used as a food additive for their health-promoting effects. Health benefits are a result of, for example, production of nutrients and/or co-factors by the probiotic, competition of the probiotic with pathogens and/or stimulation of an immune response in the subject by the probotic.

The term “probiotic composition” refers to a composition comprising a probiotic bacterium in a pharmaceutically or nutraceutically acceptable carrier that allows high cell viability after oral administration. For example, in some cases, the probiotic bacterium is lyophilized. In some cases, the probiotic bacterium is encapsulated in a gel matrix.

The term “prevent” refers to a treatment that forestalls or slows the onset of a disease or condition or reduces the severity of the disease or condition. Thus, if a treatment can treat a disease in a subject having symptoms of the disease, it can also prevent that disease in a subject who has yet to suffer some or all of the symptoms.

The term “subject” refers to any individual who is the target of administration or treatment. The subject can be a vertebrate, for example, a mammal. Thus, the subject can be a human or veterinary patient. The term “patient” refers to a subject under the treatment of a clinician e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “vector” or “construct” refers to a nucleic acid sequence capable oftransporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” mean the introduction of a nucleic acid, e.g., an expression vector, into a recipient cell including introduction of a nucleic acid to the chromosomal DNA of said cell.

By “isolated nucleic acid” or “purified nucleic acid” is meant DNA that is free of the genes that, in the naturally-occurring genome of the organism from which the DNA of the invention is derived, flank the gene, The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Fructose-Asparagine is a Primary Nutrient During Growth of Salmonella in the Inflamed Intestine

Results

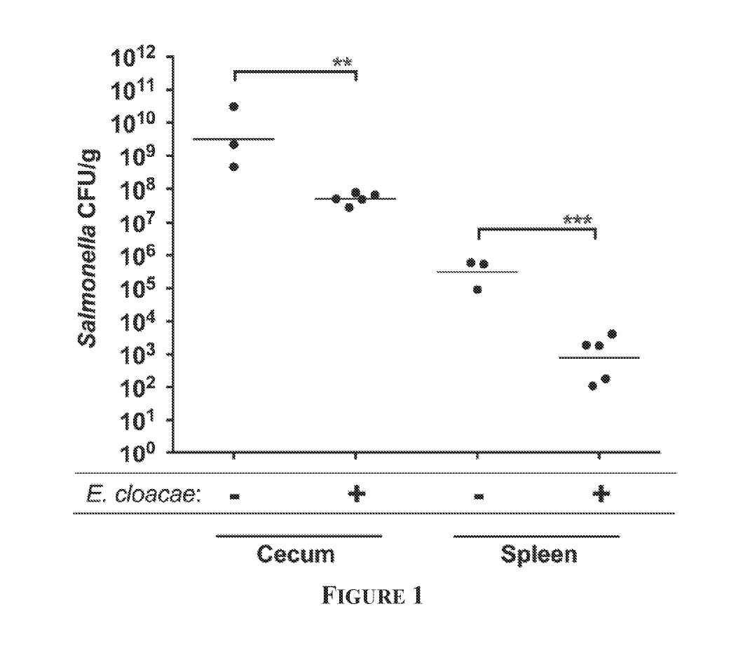

The fructose-asparagine (F-Asn) utilization system was discovered during a genetic screen designed to identify novel microbial interactions between Salmonella and the normal microbiota. Transposon site hybridization (TraSH) was used to measure and compare the relative fitness of Salmonella transposon insertion mutants after oral inoculation and recovery from the cecum of two types of gnotobiotic mice, differing from each other by a single intestinal microbial species [Chaudhuri R R, et al. (2009) PLoS Pathog 5:e1000529; Santiviago C A, et al. (2009) PLoS Pathog 5:e1000477; Lawley T D, et al. (2006) PLoS Pathog 2:e11; Badarinarayana V, et al. (2001) Nat Biotechnol 19:1060-1065; Sassetti C M, et al. (2001) Proc Natl Acad Sci USA 98:12712-12717; Goodman A L, et al. (2009) Cell Host Microbe 6:279-28]. The two types of mice were germ-free and ex-germ-free colonized by a single member of the normal microbiota, Enterobacter cloacae. E. cloacae was chosen because it is a commensal isolate from laboratory mice, easily cultured, genetically tractable, and it protects mice against Salmonella infection (FIG. 1). In total, five genes conferred a greater fitness detect in the mice containing Enterobacter than in the germ-free mice (Table 1).

TABLE 1
Genes that are differentially required in germ-free mice and
ex-germ-free mice monoassociated with Enterobacter cloacae.

| | | | Germ- | Enterobacter | |
|---|---|---|---|---|---|
| | | | free | monoassociated | |
| Locus tag^{a} | Symbol | Description | mice^{b} | mice^{c} | Difference^{d} |
| STM14_2365 | sirA | response regulator | 1.88 | −0.27 | −2.15 |
| STM14_3566 | barA | hybrid sensory | 1.09 | −0.55 | −1.64 |
| | | histidine kinase | | | |
| STM14_4330 | fraD | putative sugar kinase | −0.07 | −1.29 | −1.22 |
| STM14_4331 | fraB | putative | 0.05 | −1.12 | −1.18 |
| | | phosphosugar | | | |
| | | isomerase | | | |
| STM14_4329 | fraA | putative transporter | −0.06 | −1.23 | −1.17 |

^{a}The locus tag is from the Salmonella serovar Typhimurium strain 14028s genome (accession number NC_016856.1)
^{b}The log2 hybridization intensity of this locus after recovery of the Salmonella library from germ-free mice.
^{c}The log2 hybridization intensity of this locus after recovery of the Salmonella library from germ-free mice that had been previously monoassociated with Enterobacter cloacae.
^{d}The difference in log2 hybridization intensity of this locus between Enterobacter monoassociated mice and germ-free mice.

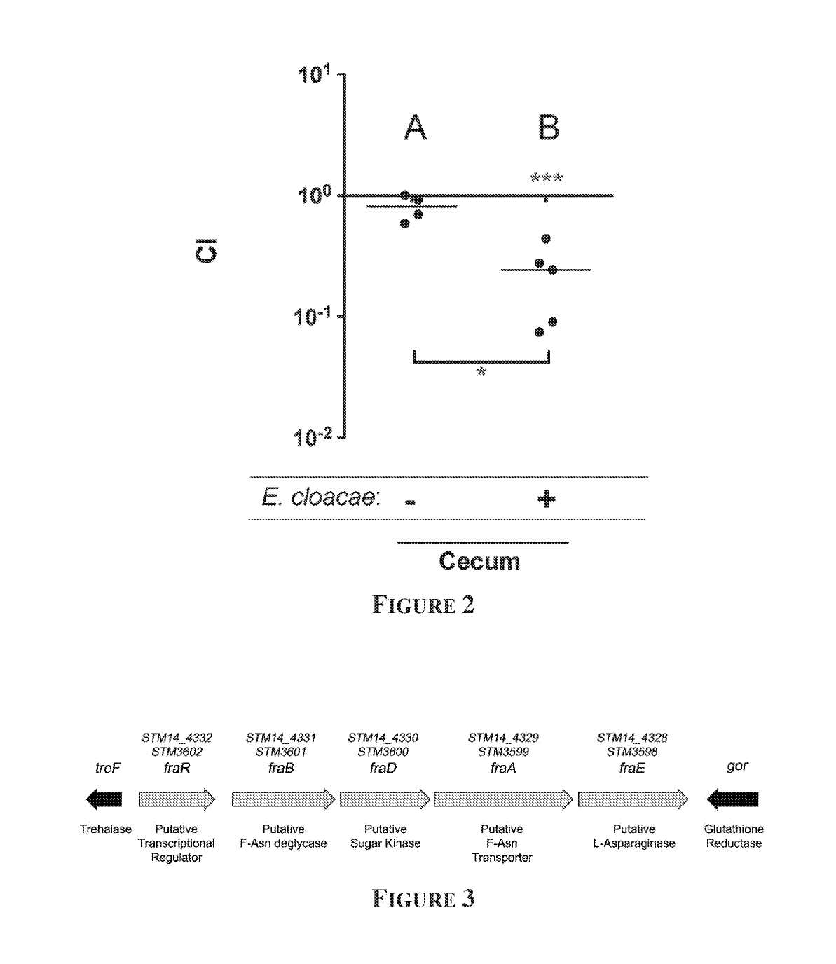

Two of these genes, barA and sirA (uvrY), encode a two component response regulator pair that is conserved throughout the c-proteobacteria [Teplitski M, et al. (2005) Research Signpost. 26 p; Romeo T, et al. (2013) Environ Microbiol 15: 313-324; Lapouge K, et al. (2008) Mol Microbiol 67:241-253]. BarA/SirA control the activity of the CsrA protein (carbon storage regulator) which coordinates metabolism and virulence by binding to and regulating the translation and/or stability of mRNAs for numerous metabolic and virulence genes including SPI1, SPI2, and glgCAP (glycogen biosynthesis) [Romeo T, et al. (2013) Environ Microbiol 15: 313-324; Lawhon S D, et al. (2003) Mol Microbiol 48:1633-1645; Marti'nez L C, et al. (2011) Mol Microbiol 80:1637-1656]. To confirm the fitness phenotype of the BarA/SirA regulatory system, competition experiments were performed in which wild-type Salmonella was mixed in a 1:1 ratio with an isogenic sirA mutant and inoculated orally into germ-free mice and ex-germ-free mice colonized by Enterobacter. The results of TraSH analysis suggested that the sir/mutant would be at a greater growth disadvantage in Enterobacter mono-associated mice than in germ-free mice (Table 1). Results of the competition experiment confirmed this prediction (FIG. 2).

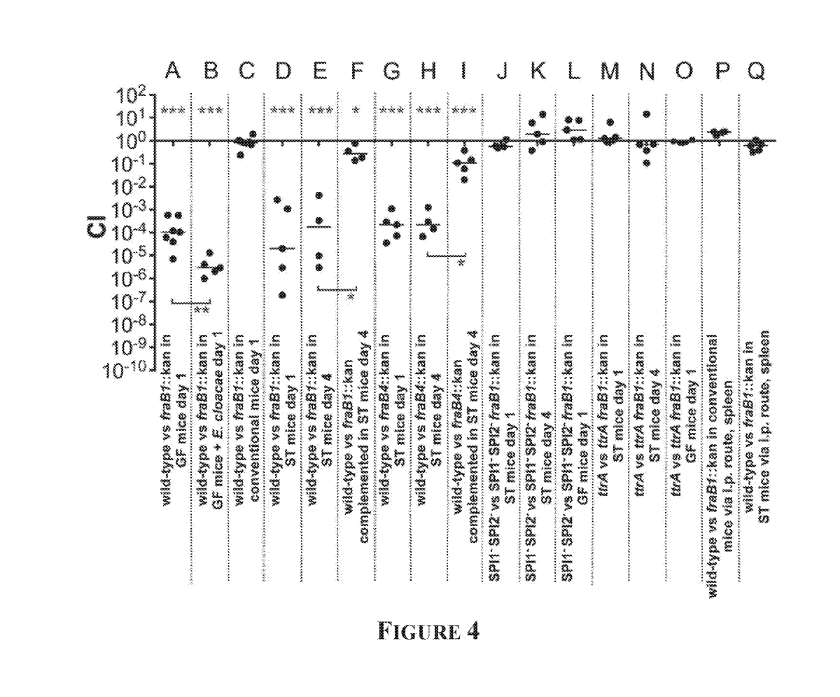

The other three genes identified by TraSH analysis had not been characterized previously, and are located together in a putative operon. Genome annotation suggested that they encode a C4 dicarboxylate transporter, a sugar kinase, and a phosphosugar isomerase (FIG. 3). A putative asparaginase lies at the end of the operon, and a separate gene upstream of the operon encodes a putative transcriptional regulator of the GntR family. These genes are not present in E. coli and appear to represent a horizontal acquisition inserted between the gor and treF genes at 77.7 centisomes of the Salmonella 14028 genome (ORFs STM14_4328 to STM14_4332). These genes are named fraBDAE and fraR for reasons to be described below. A fra B1::kan mutation was constructed and tested for fitness in germ-free and Enterobacter colonized mice using 1:1 competition assays against the wild-type Salmonella. The TraSH results suggested that this locus would exhibit a differential fitness phenotype in germ-free mice and Enterobacter mono-associated mice. Indeed, disruption of the fra locus caused a severe fitness defect in germ-free mice and a more severe defect in Enterobacter-colonized mice (FIGS. 4A, B).

The Fra Locus Confers a Fitness Advantage During Inflammation and Anaerobic Respiration

Competition experiments between wild-type and the fraB1::kan mutant were performed as described above using conventional mice (with normal microbiota) and mice treated orally with streptomycin (strep-treated) one day earlier to disrupt the microbiota (FIGS. 4C, D, E). Conventional mice do not become inflamed from Salmonella, while strep-treated mice (or germ-free) do become inflamed [Stecher B, et al. (2007) PLoS Biol 5:2177-2189; Winter S E, et al. (2010) Nature 467:426-429; Thiennimitr P, et al. (2011) Proc Natl Acad Sci USA 108:17480-17485; Barthel M, et al. (2003) Infect Immun 71:2839-2858; Woo I I, et al. (2008) PLoS ONE 3:e1603; Garner C D, et al. (2009) Infect Immun 77:2691-2702; Kaiser P, et al. (2012.) Immunol Rev 245:56-835]. Disruption of the fra locus caused no fitness defect in conventional mice, but caused a severe defect in the strep-treated mice at one and four days post-infection (FIGS. 4C, D, E). The phenotype in strep-treated mice was confirmed by complementation (FIG. 4F). It is expected that the fraB1::kan mutation is polar on the remainder of the fraBDAE operon. Therefore, the fraB1::kan mutation was complemented with a low copy number plasmid encoding the entire fra island (FIG. 4F). The phenotype was confirmed again using a separately constructed mutation, fraB4::kan, and complementation (FIGS. 4G, H, I). In both instances, greater than 99% of the phenotype was restored (FIGS. 4F, I).

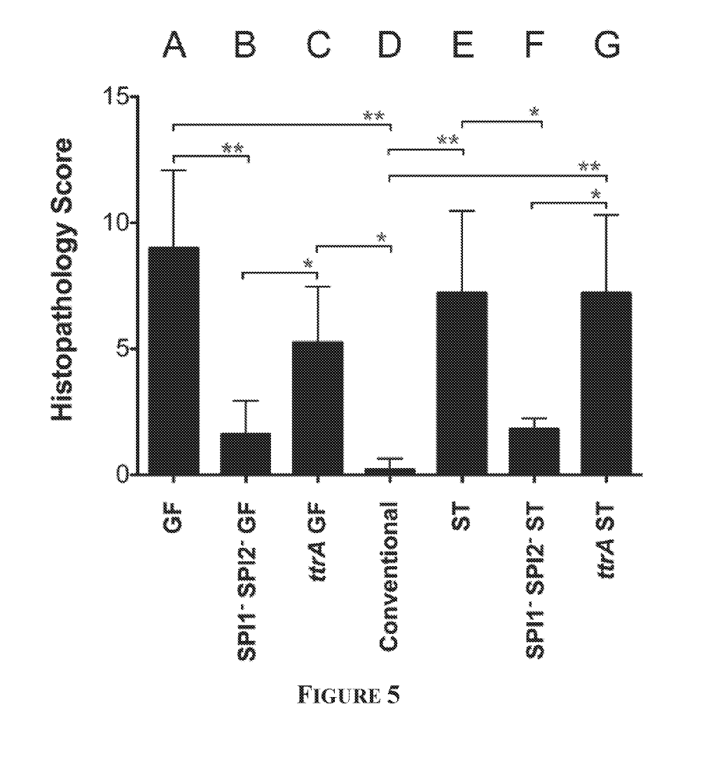

The observation of a phenotype in germ-free and strep-treated mice, but not conventional mice, suggested that Salmonella might require inflammation in order to acquire or utilize the fra-dependent nutrient source. It is known that inflammation causes the accumulation of tetrathionate in the lumen, a terminal electron acceptor that allows Salmonella to respire anaerobically [Winter S E, et al. (2010) Nature 467:426-429]. Histopathology results confirmed that infection with Salmonella caused inflammation in the germ-free and strep-treated mice, but not in the conventional mice (FIGS. 5A, 5D, 5E). To test whether Salmonella must induce inflammation for fra to affect the phenotype, the competition experiments were repeated in a Salmonella genetic background lacking SPI1 and SPI2, so that both the wild-type and the fra mutant would be defective for induction of inflammation. The severe fitness phenotype of the fra mutant was not observed in these strains (FIGS. 4J-4L) and histopathology results confirmed that inflammation was indeed low during these experiments (FIGS. 5B, 5F).

To test whether tetrathionate respiration was required for use of the fra-dependent nutrient source, the competition experiments were repeated in a ttrA mutant background. TtrA is part of a tetrathionate reductase, which is required for the utilization of tetrathionate as a terminal electron acceptor during anaerobic respiration [Winter S E, et al. (2010) Nature 467:426-429; Price-Carter M, et al. (2001) J Bacteriol 183:2463-24751. As in the SPI1 SPI2 background, there was no phenotype of a fra mutant in a ttrA mutant background indicating that Salmonella must be able to respire using tetrathionate to gain advantage from the fra locus (FIGS. 4M-4O). Histopathology results confirmed the presence of moderate inflammation during these experiments (FIGS. 5C, 5G).

To determine if the fra locus is required during the systemic phase of disease, competition experiments were performed between the wild-type and fra mutant after intraperitoneal inoculation of conventional or strep-treated mice, with bacterial recovery from the spleen. The fra mutant had no fitness defect during systemic infection (FIGS. 4P, 4Q).

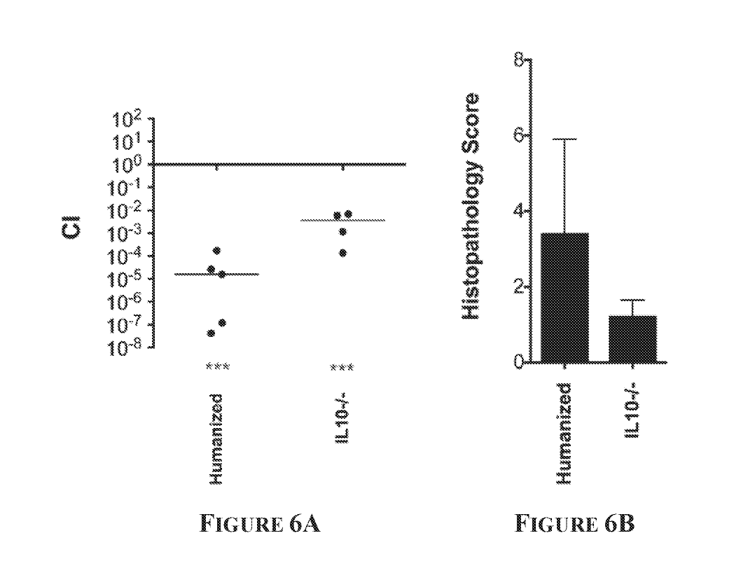

So far, we the fra phenotype has been seen in C57BL/6 mice, which are mutated at the Nramp1 locus, and this required that the mice be either germ-free or strep-treated so that Salmonella could induce inflammation. Ideally, the significance of the fra locus should be determined in a model that is not mutated and does not require strep-treatment or a germ-free status. Humans with a complete microbiota are quickly inflamed by Salmonella infection while conventional mice are not, and more recently it was discovered that germ-free mice colonized with human fecal microbiota (“humanized” mice) become inflamed from Salmonella infection without disturbance of the gut microbiota by streptomycin [Chung H, et al. (2012) Cell 149:1578-1593]. Therefore, germ-free Swiss Webster mice, which are Nramp1^{+/+}, were humanized with human feces obtained from a healthy adult donor from the Ohio State University fecal transplant center. Competition experiments were then performed between wild-type and fra mutant Salmonella in these mice. Histopathology results confirmed the presence of mild inflammation during these experiments and the fra locus had a greater than 10,000-fold fitness phenotype (FIG. 6).

IL10 knockout mice were used as another method to facilitate Salmonella-induced inflammation without using streptomycin [Stecher B, et al. (2007) PLoS Biol 5:2177-2189]. Histopathology results indicated that, unexpectedly, there was not very much inflammation in these mice by day 3 post-infection although the fra locus still had a modest fitness phenotype (greater than 100-fold) (FIG. 6). The phenotypes of the fra locus in IL10 knockout mice and in the humanized Swiss Webster mice demonstrate that the fra phenotype is not limited to germ-free or streptomycin-treated mice.

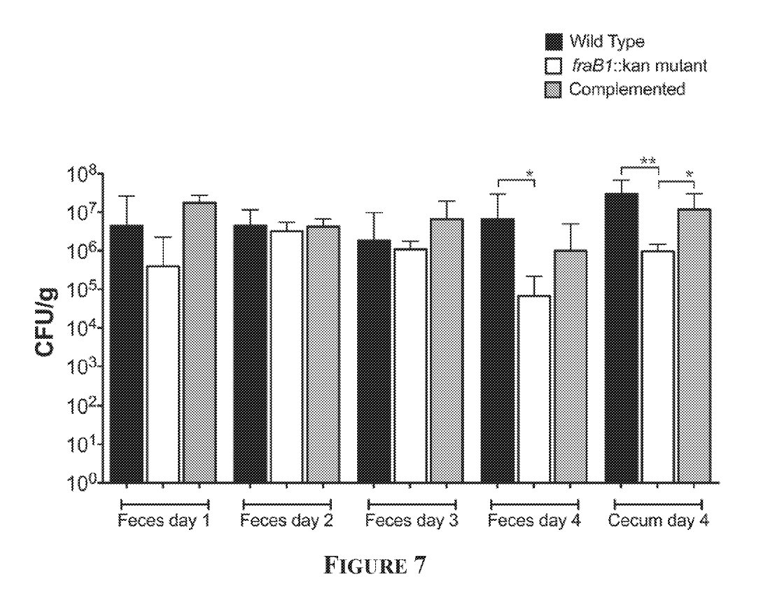

Finally, to test for the possibility that these severe fra mutant phenotypes were the result of interaction between the wild-type and fra mutant during infection, experiments were performed in which strep-treated C57BL/6 Nramp1^{+/-} heterozygous mice were infected separately with the wild-type, the fra mutant, or the complemented fra mutant. The strains were quantitated in the feces each day post-infection for four days at which point the mice were sacrificed and the strains were quantitated in the cecum. The fra mutant was recovered in 30-fold lower numbers than wild-type on the fourth day in the feces and 98-fold lower in the cecum (FIG. 7). This defect was restored by complementation with the fra locus on a plasmid in the cecum, while in the feces the restoration did not reach statistical significance (FIG. 7).

The Fra Locus is Required for Growth on Fructoseasparagine (F-Asn)

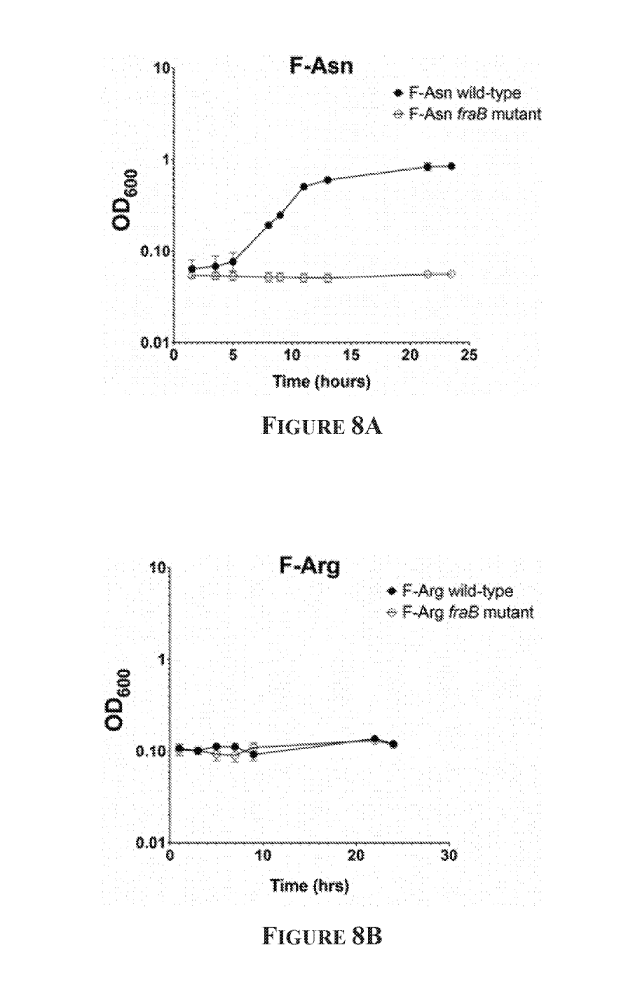

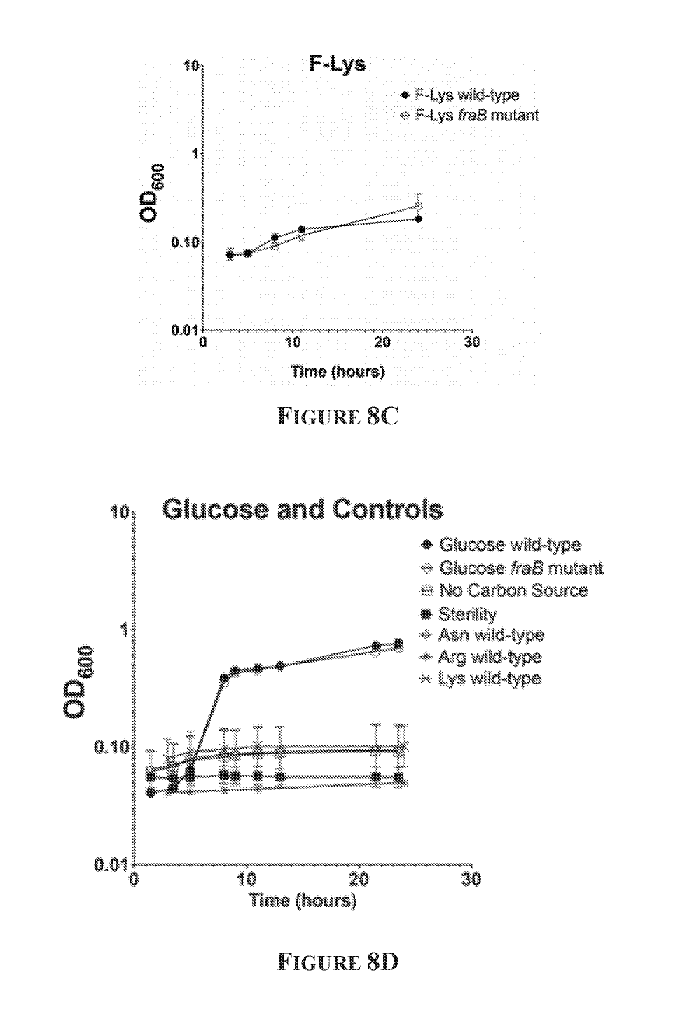

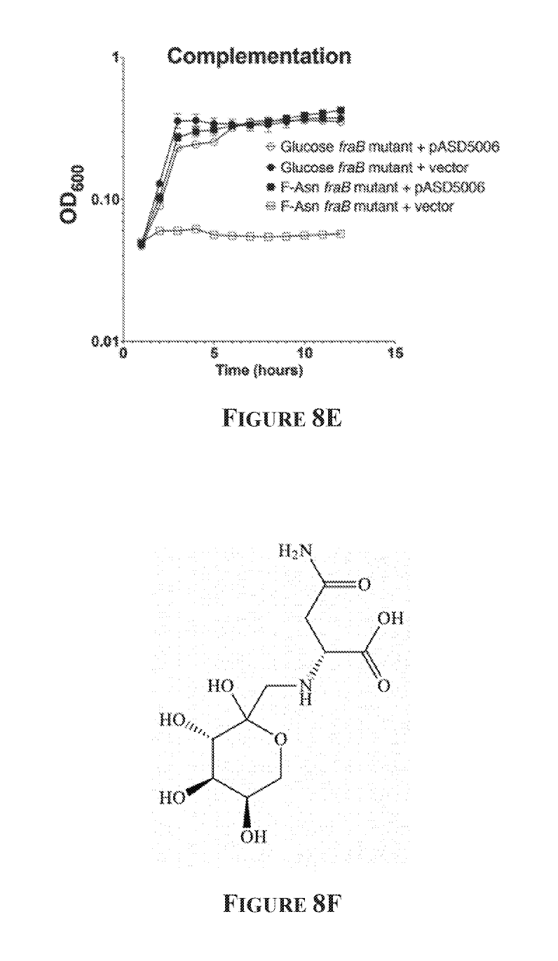

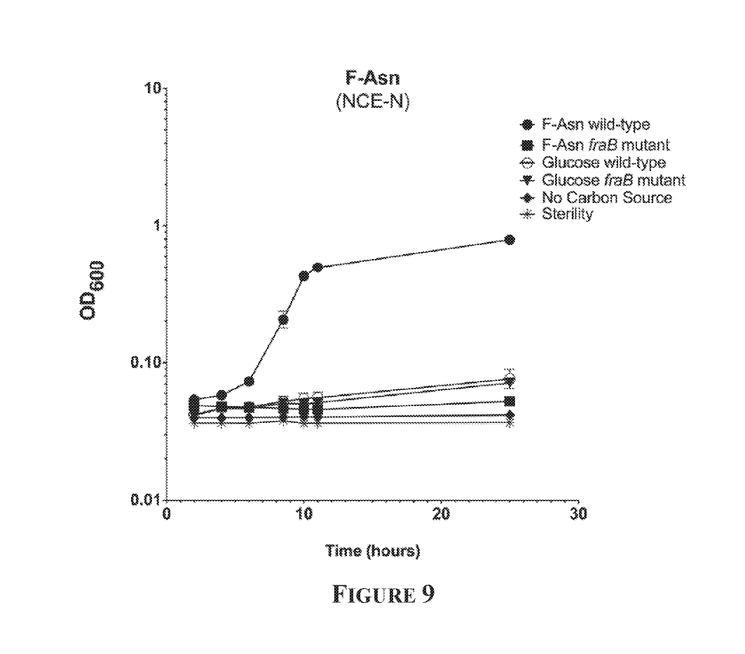

FraA is homologous to the Dcu family of dicarboxylate transporters. However, authentic dicarboxylate acquisition loci do not encode a sugar kinase or phosphosugar isomerase. Furthermore, none of the dicarboxylates that we tested (malate, fumarate or succinate) provided a growth advantage to the wildtype strain vs. a fraB1::kan mutant, suggesting that they are not substrates of the Fra pathway. BLAST searches using the entire operon revealed that the closest homolog is the frl operon of E. coli, although the frl operon is at a different location within the genome and does not encode an asparaginase (and the Salmonella fra locus does not encode a frlC homolog). The products of the E. coli frl operon transport and degrade the Amadori product fructose-lysine (F-Lys) [Wiame E, et al. (2004) Biochem J 378:1047-1052; Wiame E, et al. (2002) J Biol Chem 277:42523-425291 Amadori products most often result from a spontaneous reaction between a carbonyl group (often of glucose, although numerous other compounds can also react) and an amino group of an amino acid in vivo, and are then referred to as non-enzymatic glycation products [Zhang Q, et al. (2009) J Proteome Res 8:754-769; Tessier F J (2010) Pathol Biol 58:214-219]. With F-Lys and fructose-arginine (F-Arg) this can happen with the free amino acid, or the side groups of the lysine and arginine residues of a protein. In contrast, fructose-asparagine (F-Asn) can only result from reaction of glucose with the alpha amino group of free asparagine or the N-terminal asparagine of a protein. Three different Amadori products, F-Lys, F-Arg, and F-Asn, were synthesized and used as sole carbon sources during growth experiments. The preparations were free of glucose but contained some free amino acid. However, control experiments demonstrated that Salmonella was unable to grow on any of the three amino acids alone, so these contaminants are inconsequential (FIG. 8D). Salmonella was unable to grow on F-Arg, and grew slowly and with low yield on F-Lys (FIGS. 8B, 8C). The growth on F-Lys was independent of the fra locus. In contrast, Salmonella grew as well on F-Asn as on glucose, and growth on F-Asn was dependent upon the fra locus (hence the name fra, for fructose-asparagine utilization) (FIG. 8A). A commercial source of F-Asn was obtained and it also allowed Salmonella to grow in a fra-dependent manner (structure shown in FIG. 8F). Complementation of the fraB1::kan mutant with a plasmid encoding the fra island restored the ability of the mutant to grow on F-Asn (FIG. 8E). In addition to serving as a sole carbon source, F-Asn, also served as sole nitrogen source (FIG. 9).

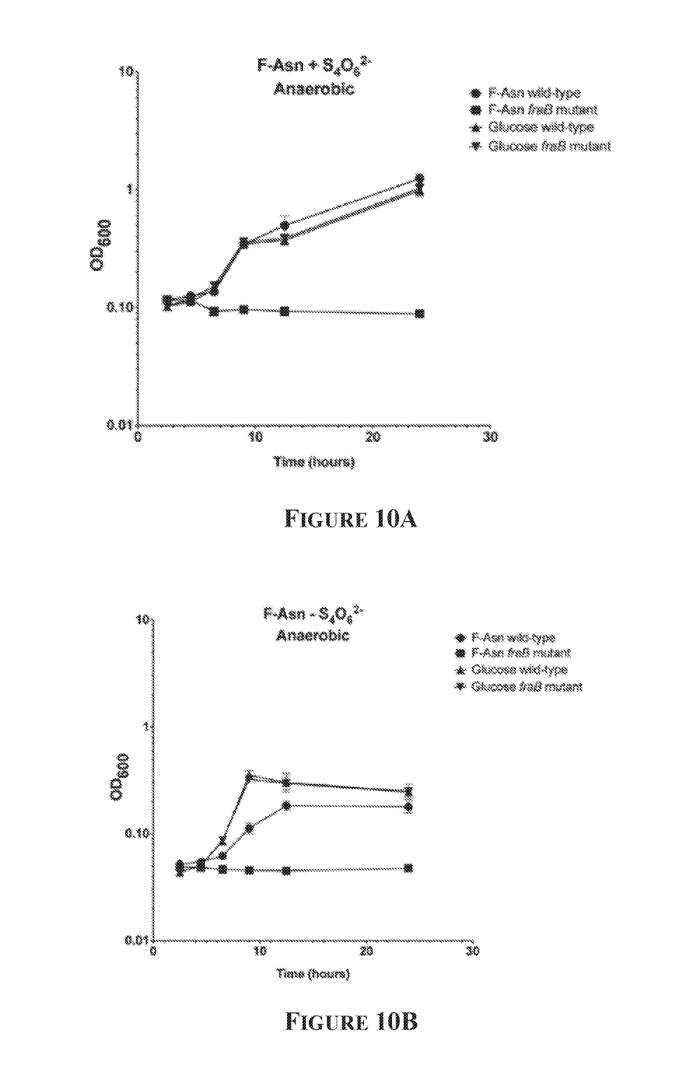

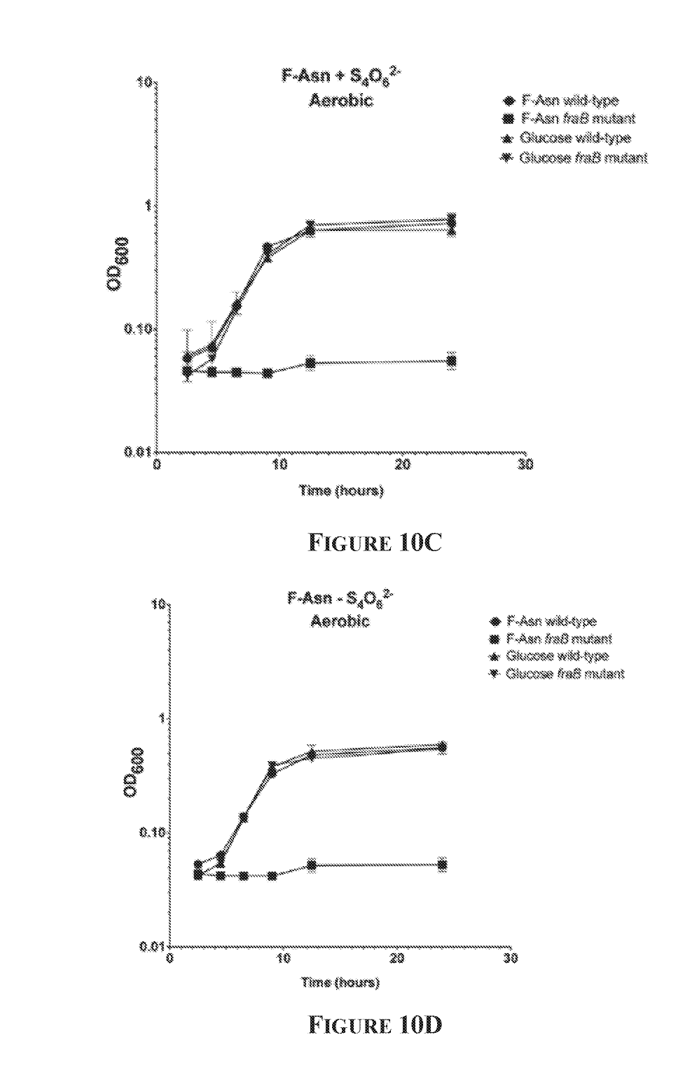

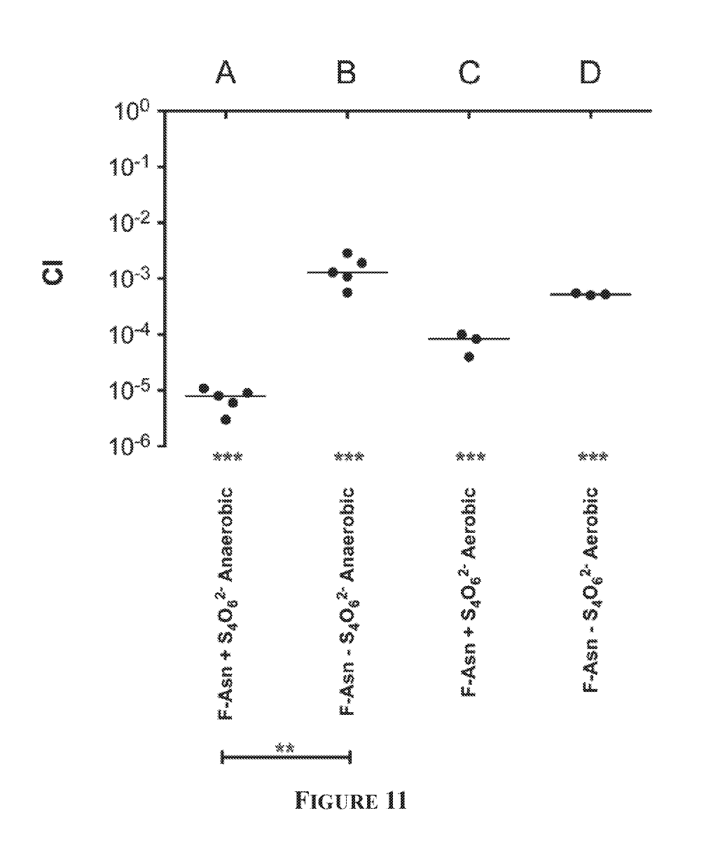

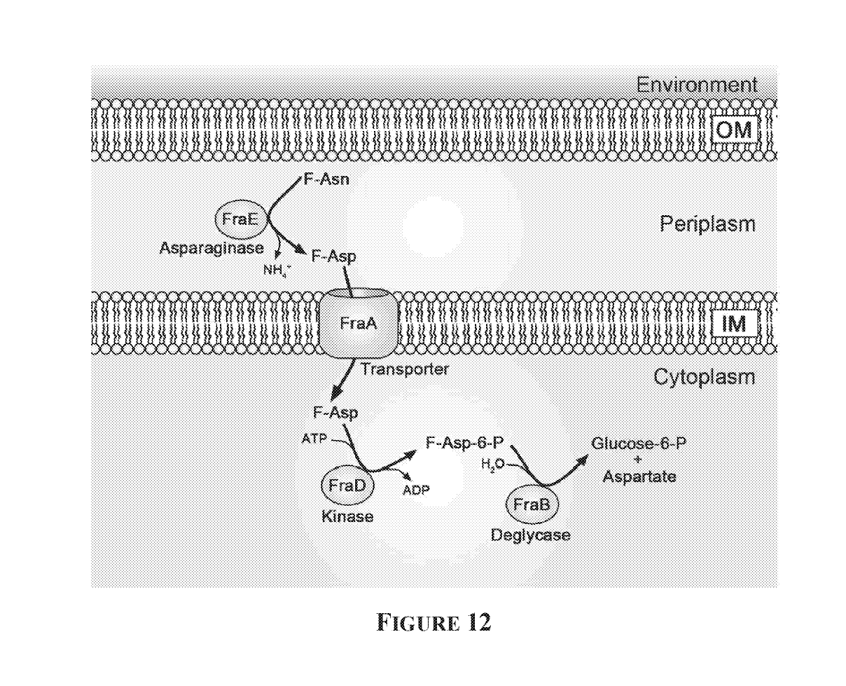

Growth with F-Asn was tested under aerobic and anaerobic conditions in the presence or absence of the terminal electron acceptor tetrathionate (FIG. 10). The F-Asn was utilized under all conditions, but respiratory conditions were superior with a doubling time of 1.6+/−0.1 hours aerobically with tetrathionate, 2.0+/−0.3 hours aerobically without tetrathionate, 1.9+/−0.1 hours anaerobically with tetrathionate, and 2.9+/−0.4 hours anaerobically without tetrathionate. Competition experiments in which the wild-type and fraB1::kan mutant were grown in the same culture were performed in minimal medium containing FAsn. As expected, the mutant was severely attenuated during aerobic and anaerobic growth, and in the presence or absence of tetrathionate (FIG. 11). The attenuation was most severe during anaerobic growth in the presence of tetrathionate.

Materials and Methods

Bacterial Strains and Media

Bacteria were grown in Luria-Bertani (LB) broth or on LB agar plates (EM Science) unless otherwise noted. The minimal medium used was NCE (no carbon E) containing trace metals [Price-Carter M, et al. (2001) J Bacteriol 183:2463-2475]. Chloramphenicol (cam), streptomycin (strep), or kanamycin ((pan) were added at 30, 200, or 60 mg/ml, respectively, when appropriate. Fructose-asparagine was either synthesized or purchased from Toronto Research Chemicals, catalog #F792525, Anaerobic growth was performed in a Bactron 1 anaerobic chamber containing 90% N2, 5% CO₂, and 5% H₂ (Shel Lab). Strains used are described in Table 2. Enterobacter cloacae strain MD400 was isolated in by plating fecal samples from a conventional BALB/c mouse onto LB agar plates. This particular isolate was chosen because it is easy to culture and genetically manipulable (the strain can be electroporated, maintains ColE1-based plasmids, and can act as a recipient in RP4-mediated mobilization of a suicide vector used to deliver mTn5-luxCDABE, not shown). The species identification was performed using a Dade Microscan Walkaway 96si at the Ohio State University medical center. Additionally, genomic DNA sequences have been obtained that flank mTn5-luxCDABE insertions in JLD400 and these DNA sequences match the draft genome sequence of E. cloacae NCTC 9394.

TABLE 2
Bacterial strains and plasmids.

| Strain or | |
|---|---|
| plasmid | Genotype or description |
| 14028 | wild-type Salmonella enterica serovar Typhimurium |
| ASD6000 | MA59 fraB1::kan + pASD5006 (amp^{r}, fraR⁺ fraBDAE⁺) |
| ASD6010 | MA59 fraB1::kan + pWSK29 (amp^{r}) |
| ASD6040 | CS1032 fraB4::kan + pASD5006 (amp^{r}) |
| ASD6090 | IR715 + pWSK29 (amp^{r}) |
| IR715 | 14028 nal^{r} |
| JLD400 | wild-type Enterobacter cloacae isolated from a |
| | laboratory mouse |
| JLD1214 | 14028 IG(pagC-STM14_1502)::cam |
| MA43 | IR715 phoN1::aadA |
| MA45 | IR715 sirA2::kan |
| MA59 | IR715 fraB1::kan |
| CS1032 | IR715 fraB4::kan |
| MA4301 | 14028 Δ(avrA-invH)1 ssaK::kan |
| MA4310 | MA43 ttrA1::cam |
| MA5900 | 14028 Δ (avrA-invH)1 ssaK::kan fraB1::cam |
| MA5910 | IR715 fraB1::kan ttrA1::cam |
| pASD5006 | pWSK29 fraRBDAE + amp^{r} |
| pWSK29 | pSC101 cloning vector amp^{r} |
| pCP20 | cI857 δPR-flp pSC101 oriTS amp^{r} cam^{r} |
| pKD3 | FRT-cam-FRT oriR6K amp^{r} |
| pKD4 | FRT-kan-FRT oriR6K amp^{r} |

Salmonella Mutant Library

A transposon mutant library was constructed in S. enterica serovar Typhimurium strain 14028. EZ-Tn5 <T7/kan> transposomes from Epicentre Technologies were delivered to Salmonella by electroporation. This transposon encodes kanamycin resistance and has a T7 RNA Polymerase promoter at the edge of the transposon pointed outward. The resulting library contains between 190,000 and 200,000 independent transposon insertions and is referred to as the JLD200k library. The insertion points of this library have been determined previously by next-generation sequencing [Canals R, et al. (2012) BMC Genomics 13:212]. It is estimated that approximately 4400 of the 4800 genes in the Salmonella genome are non-essential with regard to growth on LB agar plates [Canals R, et al. (2012) BMC Genomics 13:212]. Therefore, the JLD200k library is saturated with each gene having an average of 43 independent transposon insertions.

Construction of Mutations

A FRT-kan-FRT or FRT-cam-FRT cassette, generated using PCR with the primers listed in Table 3 and pKD3 or pKD4 as template, was inserted into each gene of interest (replacing all but the first ten and last ten codons) using lambda Red mutagenesis of strain 14028+pKD46 followed by growth at 37° C. to remove the plasmid [Datsenko K A, et al. (2000) Proc Natl Acad Sci USA 97:6640-6645]. A temperature sensitive plasmid encoding FLP recombinase, pCP20, was then added to each strain to remove the antibiotic resistance marker [Datsenko K A, et al. (2000) Proc Natl Acad Sci USA 97:6640-6645]. The pCP20 plasmid was cured by growth at 37° C. A fraB4::kan mutation was constructed using primers BA2552 and BA2553 (Table 3). A FRT-cam-FRT was placed in an intergenic region downstream of pagC using primers BA1561 and BA1562 (deleting and inserting between nucleotides 1342878 and 1343056 of the 14028 genome sequence (accession number NC_016856.1) (Table 3).

TABLE 3
Oligonucleotides used.

| Gene | Primer | | |
|---|---|---|---|
| targeted | name | Description | Sequence |
| pagC | BA1561 | Used for lambda | CTTCTTTACCAGTGA |
| | | red mutagenesis | CACGTACCTGCCTGT |
| | | in which the cat | CTTTTCTCTTGTGTA |
| | | (cam^{r}) gene was | GGCTGGAGCTGCTTC |
| | | placed downstream | G |
| | | of pagC in a | (SEQ ID NO: 11) |
| pagC | BA1562 | neutral site | CGAAGGCGGTCACAA |
| | | using pKD3 as | AATCTTGATGACATT |
| | | PCR template. | GTGATTAACATATGA |
| | | | ATATCCTCCTTAG |
| | | | (SEQ ID NO: 12) |
| fra | BA2228 | Used for ampli- | CGCAGAATCTATCCG |
| island | | fying the fra | TCCGACAACGAAC |
| | | island and | (SEQ ID NO: 13) |
| fra | BA2229 | cloning it into a | GCAGGTTAAGGCTCT |
| island | | complementation | CCGTAAAGGCCAATC |
| | | vector, resulting | (SEQ ID NO: 14) |
| | | in pASD5006. | |
| fraB | BA2552 | Used for lambda | CCTGATGTAATTAAT |
| | | red mutagenesis | ATTCCACTTTCCACA |
| | | in which the aph | TATAGCGGCGCATAT |
| | | (kan^{r}) gene was | GAATATCCTCCTTAG |
| | | placed within the | (SEQ ID NO: 15) |
| fraB | BA2553 | fraB gene using | AGAGGAAAGCATGAT |
| | | pKD4 as PCR | GGGTATGAAAGAGAC |
| | | template. | AGTTAGCAATGTGTA |
| | | | GGCTGGAGCTGCTTC |
| | | | (SEQ ID NO: 16) |

Animals

Germ-free C57BL/6 mice were obtained from Balfour Sartor of the NIH gnotobiotic resource facility at the University of North Carolina and from Kate Eaton at the University of Michigan. Germ-free Swiss Webster mice were obtained from Taconic Farms. The mice were bred and maintained under germ-free conditions in sterile isolators (Park Bioservices). Periodic Gram-staining, 16 s PCR, and pathology tests performed by the Ohio State University lab animal resources department and our own laboratory were used to confirm that the mice contained no detectable microorganisms. Conventional C57BL/6 mice were obtained from Taconic Farms. C57BL/6 mice that were heterozygous for the Nramp1 gene were generated by breeding the standard Nramp1^{+/+} mice from Taconic Farms with C57BL/6 Nramp1^{+/+} mice from Greg Barton [Arpaia N, et al. (2011) Cell 144:675-688]. IL10 knockout mice (B6.129P2-IL10^{tm1Cgn}/J) were obtained from Jackson Laboratory. Germ-free Swiss Webster mice were “humanized” by intragastric inoculation of 200 μl of human feces obtained from an anonymous healthy donor from the OSU fecal transplant center.

Transposon Site Hybridization (TraSH)

The JLD200k transposon mutant library was grown in germ-free C5713126 mice in the presence or absence of E. cloacae strain JLD400. Four mice were inoculated intragastrically (i.g.) with 10⁷ cfu of Enterobacter cloacae strain JLD400 that had been grown overnight in LB shaking at 37° C. After 24 hours these mice, and an additional four germ-free mice, were inoculated with 10⁷ cfu of the JLD200k library that had been grown overnight in shaking LB kan at 37° C. Prior to inoculation of the mice, the library was spiked with an additional mutant, JLD1214, at a 1:10:000 ratio. This mutant contains a chloramphenicol resistance (camr) gene at a neutral location in the chromosome in the intergenic region downstream of pagC [Gunn J S, et al. (2000) Infect Immun 68:6139-6146]. After inoculation of mice with the spiked library, the inoculum was dilution plated to quantitate the kanamycin resistant (kanr) Salmonella library members and the camr spike strain. The remainder of the inoculum was pelleted and saved as the “input” for hybridization to microarrays. After 24 hours of infection with the JLD200k library, the mice were euthanized and organs were harvested (small intestine, cecum, large intestine, and spleen). One germ-free mouse died prior to organ harvest and was not used. All samples were homogenized and dilution plated to determine Salmonella counts. The remainder of the homogenate was added to 25 ml LB kan and grown overnight with shaking at 37° C. to recover the library members. Each culture was then pelleted and frozen as a potential “output” sample for microarray analysis. The kan^{r} and cam^{r} colony counts recovered from each organ indicated that the spike ratio of 1:10,000 was maintained in the intestinal samples but not in the spleen samples. This indicates that the library underwent a population bottleneck on the way to the spleen so microarray analysis of spleen samples would not be informative. The cecum samples were chosen for microarray analysis. There was one “input” sample for all arrays. There were seven separate “output” samples for the arrays; four from the cecums of Enterobacter-associated mice and three from germ-free mice. The output from each mouse was compared to the input on a single array. S single “in vitro” array experiment was also conducted in which the JLD200k library was grown in the presence of Enterobacter in liquid LB broth shaking at 37° C.

Genomic DNA was isolated from the input and output bacterial pellets. The purity and concentration of the DNA samples was assessed using a NANODROP spectrophotometer and the quality of the DNA was assessed via agarose gel electrophoresis. All seven samples had high quality intact genomic DNA. The DNA was digested using a restriction endonuclease (Rsa1). Labeled RNA transcripts were obtained from the T7 promoter by in vitro transcription. A two-color hybridization strategy was employed. RNA transcripts from the output samples were fluorescently labeled with Cyanine-5 (Cy5, red), while the input sample was labeled with Cyanine-3 (Cy3, green). Equal molar concentrations of the output and input sample were combined and hybridized to genome-wide tiling microarrays printed commercially by Agilent Technologies. Agilent's SUREPRINT technology employs phosphoramadite chemistry in combination with high performance Hewlett Packard inkjet technology for in situ synthesis of 60-mer oligos. Using Agilent eArray, an easy-to-use web-based application, the arrays used by Chaudhuri et al. that completely tiled both the sense and anti-sense strands of the Salmonella SL1344 genome (AMADID 015511) were synthesized [Chaudhuri R R, et al. (2009) PLoS Pathog 5:e10005291. Each slide contained 2 arrays, each array with 105,000 features, densely tiling the entire genome. The strain of Salmonella used in the experiments was 14028 and its genome sequence was only recently published (GenBank Nucleotide Accession CP001363 (complete genome) and CP001362. (plasmid)). As such, each of the 60-mer probes used by Chaudhuri et al, [Chaudhuri R R, et al. (2009) PLoS Pathog 5:e1000529] were mapped to the 14028 genome using blast, and then annotated with any open reading frames (ORFs) that the probe spanned. A total of 96,749 probes mapped to the 14028 genome, with a median gap between each probe of 35 nucleotides on both strands.

After purification, the labeled samples were denatured and hybridized to the array overnight. Microarray slides were then washed and scanned with an Agilent G2505C Microarray Scanner, at 2 min resolution. Images were analyzed with Feature Extraction 10.5 (Agilent Technologies, CA). Median foreground intensities were obtained for each spot and imported into the mathematical software package “R”, which was used for all data input, diagnostic plots, normalization and quality checking steps of the analysis process using scripts developed specifically for this analysis. In outline, the intensities were not background corrected as this has been shown to only introduce noise. The dataset was filtered to remove positive control elements and any elements that had been flagged as bad, or not present in the 14028 genome. Using the negative controls on the arrays, the background threshold was determined and all values less than this value were flagged. Finally, the Log2 ratio of output Cy5/input Cy3 (red/green) was determined for each replicate, and the data was normalized by the loess method using the LIMMA (Linear models for microarray data) package in “R” as described [Smyth G K, et al. (2003) Methods 31:265-273; Smyth G K, et al. (2003) Methods Mol Biol 224:111-136].

Complete statistical analysis was then performed in “R”. Insertion mutants where the ORF is essential for survival are selected against, and thus a negative ratio of Cy5/Cy3 (red/green) is observed in the probes adjacent to the insertion point, resulting from higher Cy3 (green) signal from the input. Conversely, insertion mutants that were advantageous to growth in the output samples would have a positive ratio, resulting from the higher Cy5 (red) signal in the output. Mutants having no effect on growth would have equal ratios in both the output and input samples (yellow).

Synthesis of Amadori Products

Three fructosyl amino acids were synthesized with asparagine, lysine, and arginine. Hodge and Fisher's review of Amadori products was consulted as an essential starting point for synthesis [Hodge J E, et al. (1963) Methods in Carbohydrate Chemistry 2:99-107] and the recent review by Mossine and Mawhinney of all aspects of fructose-amines was a treasure house of information [Mossine V V, et al. (2010) Adv Carbohydr Chem Biochem 64:291-402]. The method of Wang et al. [Wang J, et al. (2008) J Mass Spectrom 43:262-264] was found to be the most satisfactory, however reaction times cannot be standardized and excess glucose must be removed. The reaction with asparagine is slow because asparagine is sparingly soluble in methanol. By contrast, the reaction with a-Boc-lysine is fast. Arginine is an intermediate case. Previous syntheses of F-Asn include those of Stadler et al. [Stadler R H, et al. (2004) J Agaric Food Chem 52:5550-5558], Wang et al. [Wang J, et al. (2008) J Mass Spectrom 43:262-264], and Miura et at. [Miura Y, et al. (1973) Agric Biol. Chem 37:2669-2670]. The procedure of Stadler et al. [Stadler R H, et al. (2004) J Agric Food Chem 52:5550-5558] uses alkaline conditions which we thought could bring about isomerization of the sugar and racemization of the amino acid. The synthesis of Wang et al. [Wang J, et al. (2008) J Mass Spectrom 43:262-264] was developed after trying a number of different protocols described for other amino acids [Keil P, et al. (1985) Acta Chem Scand, B, Org Chem Biochem 39:191-193; Krause R, et al. (2003) Eur Food Res Technol 216:277-283; Srinivas S M, et al. (2012) J Agric Food Chem 60:1522-1527; Weitzel G, et al. (1957) Chem Ber 90:1153-1161]. Wang et al. [Wang J, et al. (2008) J Mass Spectrom 43:262-264], however, describe only a general method and asparagine presents some particular problems, the most important of which is the poor solubility of asparagine in methanol. Bisulfite was added to the reaction mixture to reduce the formation of colored by-products [Anet EFLJ (1957) Aust J Chem 10:193-197] and excess glucose was finally removed by use of a cation-exchange column according to the method of Mossine et al, [Mossine V V, et at. (1994) Carbohydr Res 262:257-270]. Using methanol alone as solvent gives the product after refluxing for 24 hr. in approximately 10-15% yield together with recovery of about 90% of the asparagine. Although the yield is low, the starting materials are inexpensive, and the insolubility of asparagine has the advantage that F-Asn, which is quite soluble in methanol, emerges from the ion exchange column almost free of asparagine. This gave a free-flowing off-white non-hygroscopic solid. The ¹H-NMR spectrum is complex due to the equilibrating mixture of alpha- and beta-pyranose and furanose forms [Mossine V V, et al, (2010) Adv Carbohydr Chem Biochem 64:291-402], but integration of the upfield resonances due to asparagine and the downfield resonances due to the sugar are in the proper ratio. The material was also characterized by its specific rotation and infrared (IR) spectrum: [α]23_{D}−48° (c=0.1, water) (reference [Miura Y, et al. (1973) Agric Biol Chem 37:2669-2670] −40°, c=1, water); IR (Nujol): 3350,3155,1668,1633,1455,1408,1080 cm⁻¹. Compare preparations to results in [Hodge J E, et al. (1963) Methods in Carbohydrate Chemistry 2:99-107; Miura Y, et al. (1973) Agric Biol Chem 37:2669-2670].

Competition Assays

Competition assays were performed in which a mutant strain was mixed in a 1:1 ratio with an isogenic wild-type and inoculated by the intragastric (i.g.) or intraperitoneal (i.p.) route to mice. Fecal samples, intestinal sections, spleen and liver were recovered at specific times post-infection, homogenized and plated on selective plates. The wild-type and mutant strains were differentiated by antibiotic resistance. The competitive index was calculated as CI=(cfu of mutant recovered/cfu w.t. recovered)/(cfu mutant input/cfu w.t. input). If the mutant is defective compared to the wild-type it will have a CI of less than 1.

Complementation Assays

The fra island was PCR amplified from purified 14028 genomic DNA with primers BA2228 and BA2229 using Phusion polymerase (New England Biolabs). The PCR product was cloned into pPCR-Blunt (Invitrogen). The resulting clones were digested with EcoRI (New England Biolabs), run on an agarose gel and the 8.6 kbp fra fragment was gel purified (Qiagen). This purified DNA fragment was ligated into pWSK29 digested with EcoRI (NEB) using T4 DNA ligase (New England Biolabs) overnight at 4° C. The ligation reaction was transformed into DH5a and plated on LB containing ampicillin at 37° C. The resulting plasmid, pASD5006, or the vector control pWSK29, were electroporated into the appropriate strains.

Ethics Statement

All animal work was performed in accordance with the protocols approved by the Institutional Animal Care and Use Committee (OSU 2009A0035). The IACUC ensures compliance of this protocol with the U.S Animal Welfare Act, Guide for Care and Use of Laboratory Animals and Public Health Service Policy on Humane Care and Use of Laboratory Animals. Human fecal material was obtained from an anonymous healthy donor at the Ohio State University fecal transplant center in accordance with the protocol approved by the Institutional Review Board (OSU 2012H0367).

Supporting Information

Dataset S1 Transposon Site Hybridization data from germ-free mice and germ-free mice monoassociated with Enterobacter Cloacae. A normalized Log2 ratio of output/input hybridization intensity was determined for each replicate. Insertion mutants where the ORF is essential for survival were selected against, and thus yielded a negative ratio in the probes adjacent to the insertion point. Conversely, insertion mutants that were advantageous to growth in the output samples yielded a positive ratio.

### Example 2

### The Use of E. coli or Salmonella Derivatives to Prevent or Treat Salmonella Infection

Results

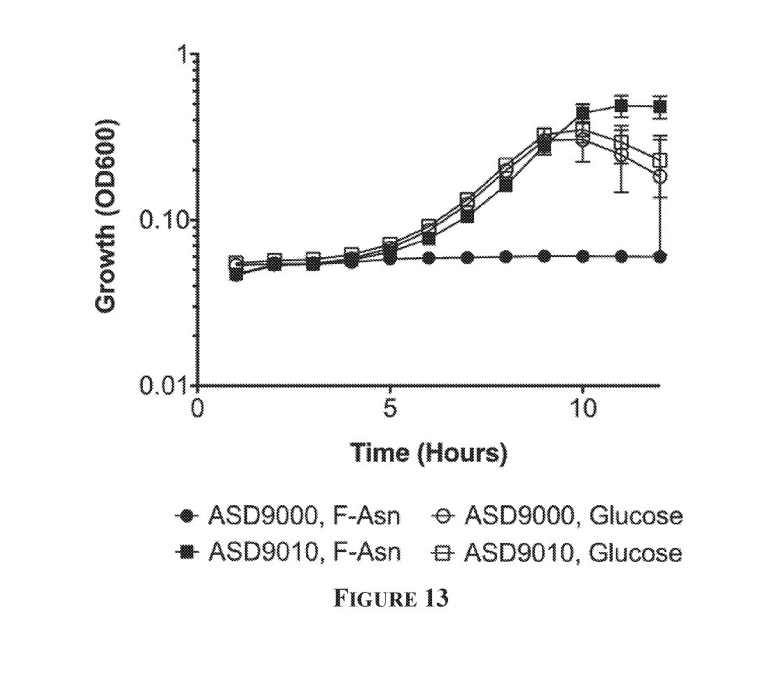

A fra mutant of Salmonella is attenuated in several murine inflammation models, suggesting that F-Asn is a nutrient that is important to Salmonella fitness in the inflamed intestine (Ali M M, et al. 2014. PLoS Pathog 10:e1004209). Therefore, adding the fra locus to a probiotic organism may enhance the ability of that organism to compete with Salmonella for F-Asn and prevent or treat Salmonella infections. To test this, the Salmonella fra locus was cloned on a low copy number plasmid and this plasmid was placed in the well characterized probiotic strain E. coli Nissle 1917 (Nissle). Nissle carrying the fra plasmid (ASD9010) was able to grow on F-Asn as sole carbon source while Nissle carrying the vector alone (ASD9000) was not (FIG. 13). Instead of adding more nutrient acquisition systems to Nissle, a mutant of Salmonella lacking SPI1 and SPI2 (ASD200) was also tested. This strain should compete with wild-type Salmonella for all nutrients without causing disease. In later experiments, a SPI1 SPI2 fra triple mutant (ASD201) was also tested to determine the fra-dependence of any observed effects. These four strains are referred to as the “probiotics” throughout this example.

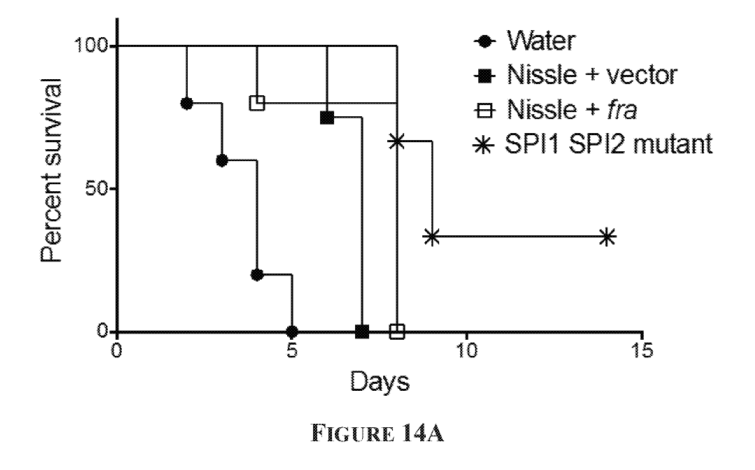

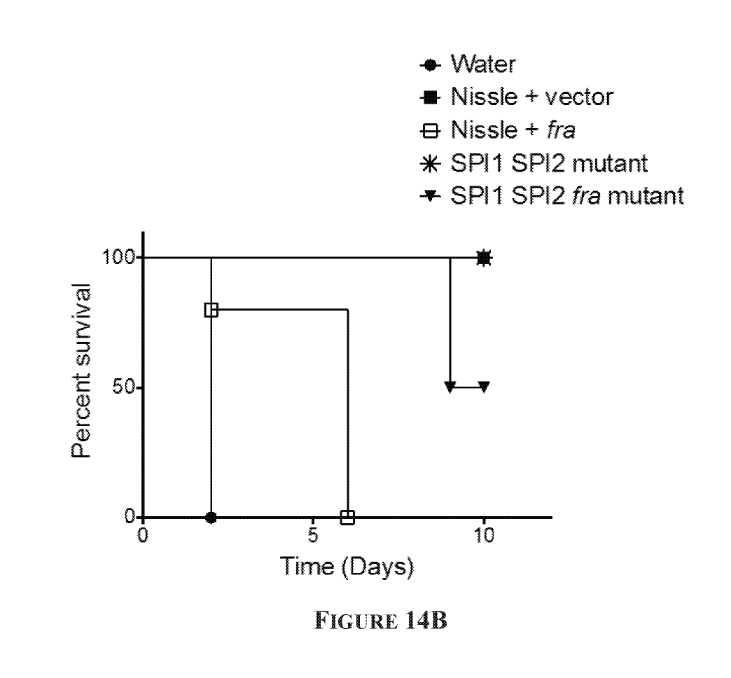

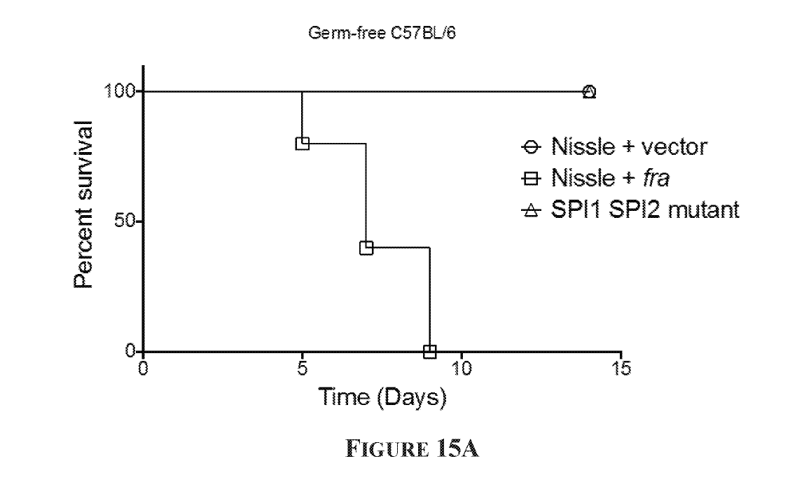

To determine if the probiotics could protect mice from wild-type Salmonella, germ-free mice were used, which have no colonization resistance. Both Swiss Webster and C57BL/6 mice were used (Nramp1^{+/+} and Nramp1^{G169D/G169D}, respectively). 10⁹ CFU of a probiotic strain or sham (water) were administered by oral gavage to groups of five mice. The following day the mice were challenged with a lethal dose of 10⁴ CFU of virulent Salmonella (strain JLD1214 which is a chloramphenicol resistant derivative of ATCC14028). In both germ-free C57BL/6 mice and in germ-free Swiss Webster mice, all of the probiotics enhanced survival (FIG. 14). Nissle+appeared slightly better than Nissle+vector in germ-free C57BL/6 mice but this was not statistically significant (P=0.075). Interestingly, Nissle+vector was highly protective in germ-free Swiss-Webster mice (100% survival), but Nissle+fra was less protective (time to death was increased over sham, but 0% survival) (P=0.004). The Salmonella SPI1 SPI2 mutant was the most protective in both types of mice, suggesting that competing for all nutrient sources is more effective than competing for a subset as is the case with Nissle. The Salmonella triple mutant (SPI1 SPI2 fra) was only used in the germ-free Swiss Webster mice. While it appeared less protective than the double mutant (SPI1 SPI2) this was not statistically significant (P=0.091).

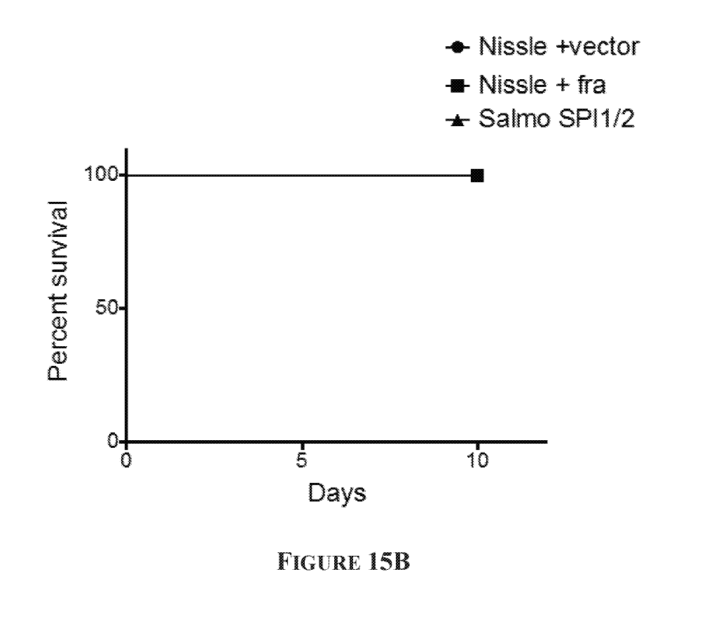

To test the safety of the probiotics, each strain was administered at a dose of 10⁹ CFU to a group of germ-free mice and mortality was monitored (FIG. 15). The Salmonella SPI1 SPI2 mutant, and the Nissle+vector, were completely safe in both types of mice (no mortality). The Nissle+fra caused no mortality in the Swiss Webster mice but caused 100% mortality in the C57BL/6 mice. This indicates that the addition of the fra locus to Nissle increased its virulence for germ-free C57BL/6 mice.

The experiments in germ-free mice revealed that the Nissle+fra strain was less effective than Nissle+vector at preventing death in germ-free Swiss-Webster mice, and it gained the ability to kill germ-free C57BL/6 mice. Thus, the ability to utilize F-Asn enhanced the virulence of Nissle. In contrast, the Salmonella SPI1 SPI2 mutant was safe and effective in protecting both C57BL/6 and Swiss Webster mice from wild-type Salmonella.

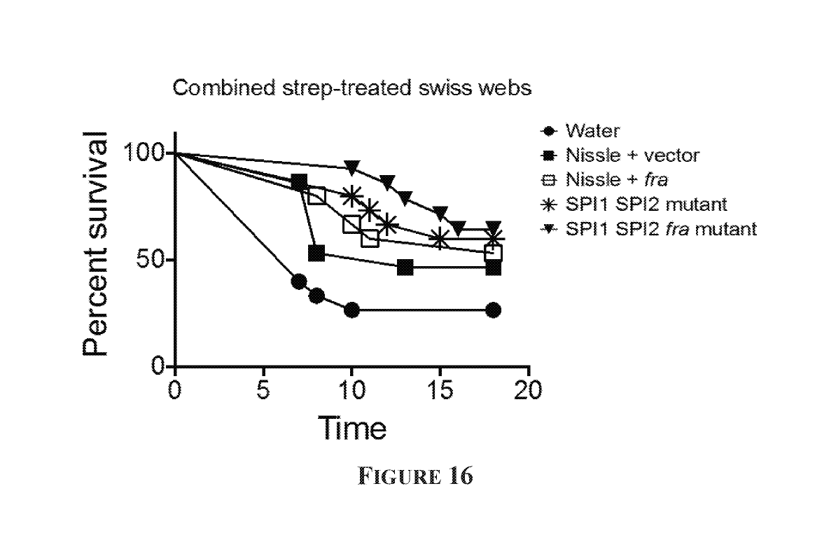

To further test the ability of these strains to protect against a lethal Salmonella infection, a strep-treated Swiss Webster mouse model was used. Mice with a normal microbiota are highly resistant to Salmonella-mediated inflammation, but treatment with streptomycin disrupts the microbiota and allows Salmonella-mediated inflammation to occur within a day of infection. Thus, in this experiment the mice were treated with streptomycin, one day later they were treated with a dose of 10⁹ CFU of a probiotic strain or sham, and one day after that they were challenged with a lethal dose of Salmonella (10⁷ CFU of JLD1214). All of the probiotic strains appeared to protect the mice from killing, except that Nissle+vector was not statistically significant (P=0.106) (FIG. 16). The protection provided by Nissle+fra was statistically different than sham, but was not different than Nissle+vector (P=0.523) making it difficult to conclude that the ability to utilize F-Asn improved the ability of Nissle to protect against Salmonella (FIG. 16). The Salmonella SPI1 SPI2 mutant and the SPI1 SPI2 fra triple mutant were both statistically different than sham, but they were not different from each other (P=0.684) indicating that protection is not dependent upon the ability to utilize F-Asn (FIG. 16).

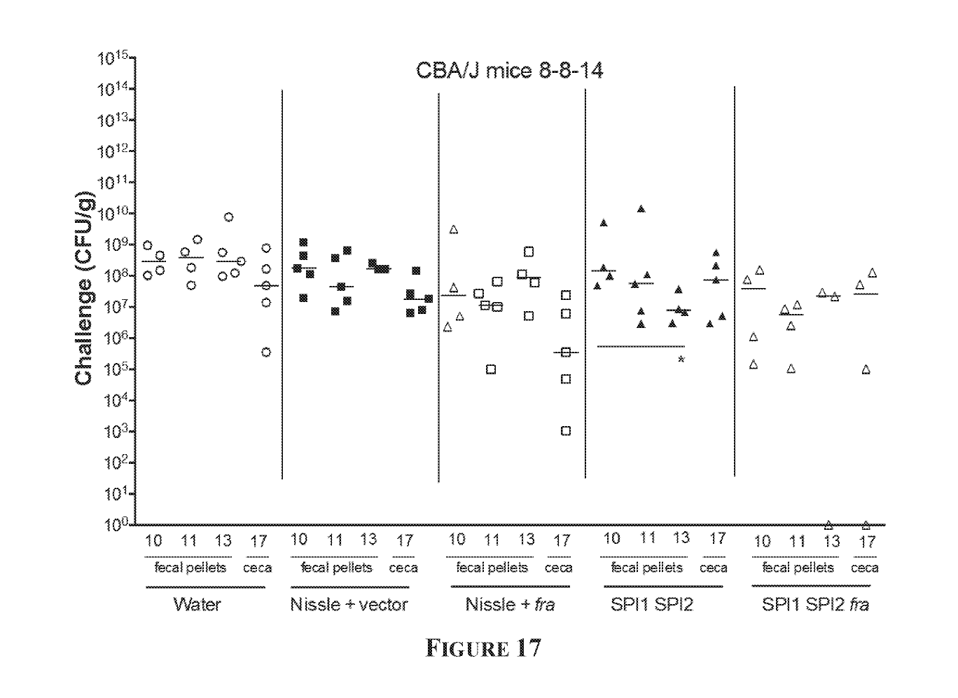

A more recent mouse model of Salmonella-mediated inflammation is the CBA/J model. These mice are Nramp1^{-/+}, tend to carry Salmonella for long periods in their intestinal tract, and become inflamed by day 10 post-infection (Lopez C A, et al. 2012. MBio 3; Rivera-Chávez F, et al. 2013. PLoS Pathog 9:e1003267). With no need for disruption of the microbiota with antibiotics, this is among the most “natural” of models. To test the ability of the probiotic strains to treat a Salmonella infection, the mice were inoculated with 10⁹ CFU of Salmonella, 10 days passed for inflammation to begin, and then the mice were treated with 10⁹ CFU of probiotic or sham. Thus, this is a therapeutic rather than a prophylactic model. Salmonella shedding in feces was measured on days 10 (just before probiotic inoculation), 11, 13, and 17 (FIG. 17). The CFU of virulent Salmonella in ceca were not reduced by any treatment compared to sham. However, in fecal samples the Nissle+fra appeared to reduce Salmonella shedding in feces by day 17, but this just missed statistical significance with a P value of 0.055. The only probiotic strain to cause a statistically significant decrease in fecal counts of virulent Salmonella compared to sham was the Salmonella SPI1 SPI2 mutant. The SPI1 SPI2 fra triple mutant was not different than sham, which might suggest that protection is fra-dependent, however it was not different than the SPI1 SPI2 mutant either (P=0.999) leaving the fra-dependence unlikely.

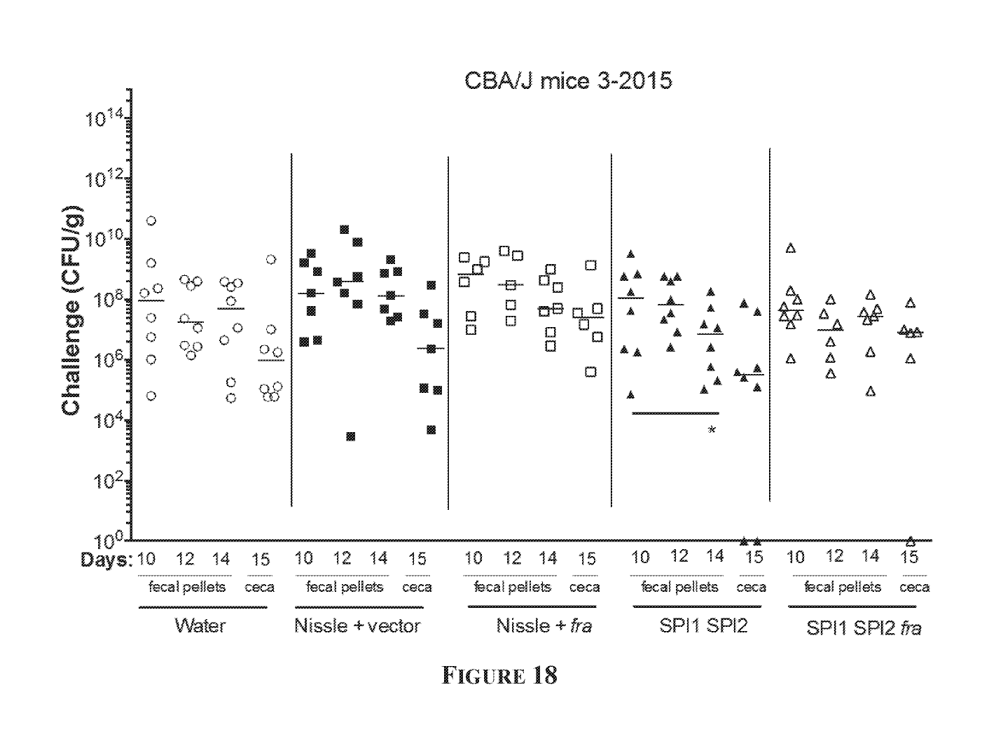

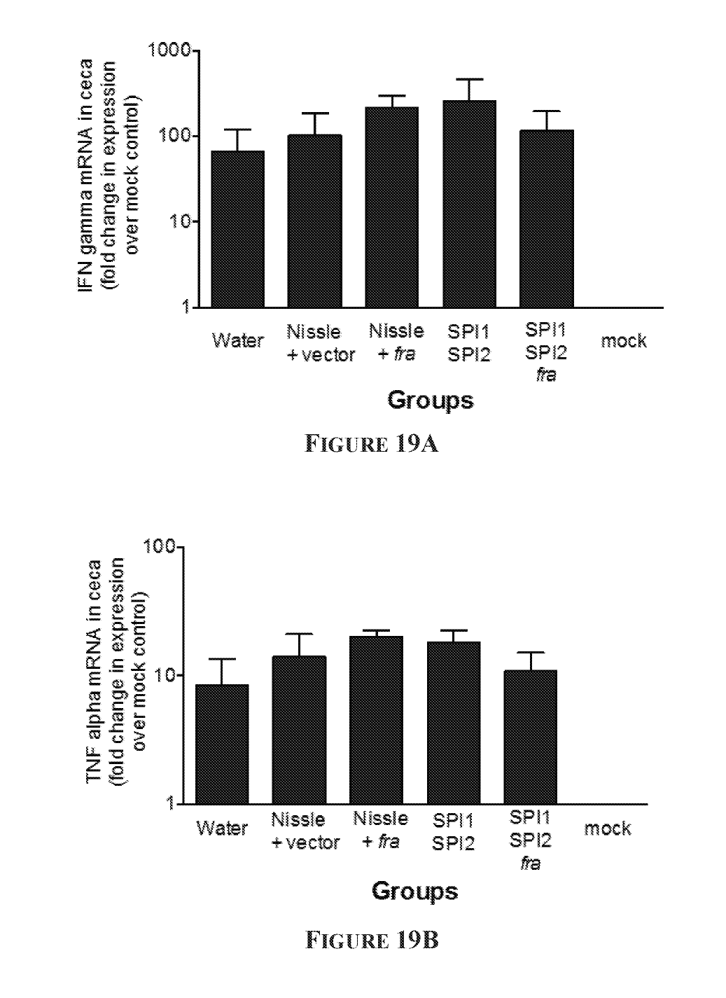

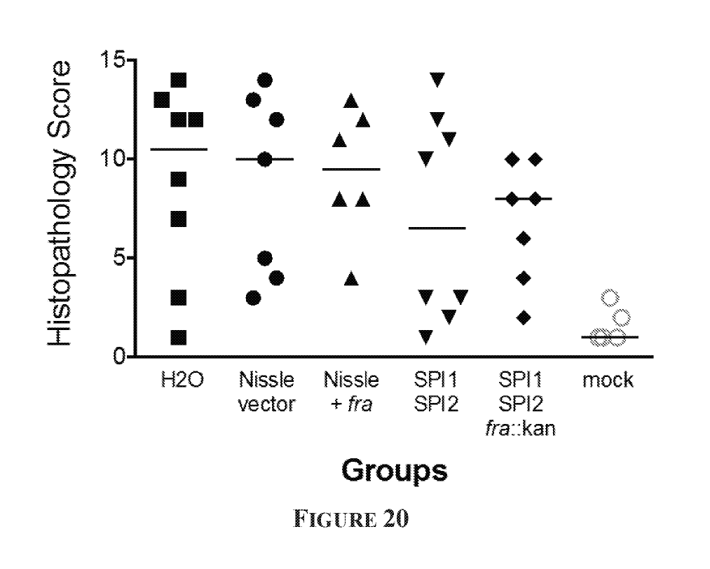

The CBA/J model was used a second time in which the number of mice per group was increased from 5 to 8, and the number of probiotic doses was increased from one to three, administered on days 10, 12, and 14 post-infection (FIG. 18). As in the previous experiment, only the SPI1 SPI2 mutant reduced the counts of virulent Salmonella compared to sham. Again, the SPI1 SPI2fra triple mutant was not different than sham suggesting that there is fra-dependence to the protection. However, the triple mutant was not different than the double (P=0.527), again leaving the fra-dependence in question. For this experiment histopathology and qRT-PCR of inflammatory markers was also performed on ceca harvested on day 15 to determine if inflammation was reduced by the probiotics. Using qRT-PCR, it was determined is that neither IFN-γ nor TNFα were reduced by treatment with the probiotics (FIG. 19). Histopathology also showed that there were no statistically significant differences between the treatment and sham groups (FIG. 20). However, the mice treated with the Salmonella SPI1 SPI2 mutant appeared to fall into two categories, with half having little or no inflammation, while the other half were highly inflamed. As a group there may be no statistically significant improvement, but for some individuals the treatment may be effective. Consistent with this, the only mice that were completely cleared of wild-type Salmonella from their cecum were two mice that had been treated with the Salmonella SPI1 SPI2 mutant, and one mouse that had been treated with the Salmonella SPI1 SPI2fra triple mutant.

Materials and Methods

Strains and media. Bacteria were grown in LB broth or on LB agar plates for routine culture (EM Science). Difco XLD agar was used for recovery of Salmonella from mice (BD). M9 minimal medium was made as described previously, and contained either 5 mM glucose or 5 mM fructose-asparagine (F-Asn) as carbon source (Miller J H. 1972. Cold Spring Harbor Laboratory, Cold Spring Harbor, N.Y.). F-Asn was synthesized as previously described (Ali M M, et al. 2014. PLoS Pathog 10:e1004209). When necessary, ampicillin (amp) or kanamycin (kan) was added to media at 200 mg/L or 60 mg/L, respectively.

Addition of the Salmonella fra locus to E. coli Nissle 1917. The low copy number plasmids, pASD5006, encoding the Salmonella strain 14028 fra locus, and the vector pWSK29, were electroporated into the E. coli dam dcm strain JM110 to decrease methylation, and then purified and electroporated into E. coli Nissle 1917 selecting on LB amp. The ability of Nissle to grow on F-Asn was confirmed by growing Nissle+pASD5006 (ASD9010) in M9 minimal medium with F-Asn as the sole carbon source compared to Nissle+pWSK29 (ASD9000) (FIG. 13). This was done in a 96-well clear bottom plate with the optical density at 600 nm recorded over an 18 hour period using a SpectraMax M5 (Molecular Devices) and SoftMax Pro 6.1 software.

Construction of a Salmonella SPI1 SPI2 mutant: Lambda Red mutagenesis was used to construct the SPI2 mutant ASD100 (Datsenko K A, et al. 2000. Proc Natl Acad Sci USA 97:6640-6645). Oligonucleotides containing 40 nucleotides of homology to either ssrB or ssaU, including 30 nucleotides of the coding region of either target were appended to sequences that bind pKD4, creating primers BA2558 and BA2559 (Datsenko K A, et al. 2000. Proc Natl Acad Sci USA 97:6640-6645). These were used to amplify the kan cassette from pKD4 using Taq DNA polymerase (NEB). The resulting PCR product, a FRT-kan-FRT cassette flanked by homology to ssrB and ssaU, was electroporated into strain 14028+pKD46 and transformants were selected on LB kan at 37° C. The insertion was verified by PCR using primers BA2582 and BA1922 (K1). This SPI2::kan mutation was transduced from ASD100 into the ΔSPI1 strain YD039 (Teplitski M, et al. 2006. Microbiology (Reading, Engl) 152:3411-3424) using phage P22HTint, creating ASD199. The antibiotic resistance marker was deleted by electroporating ASD199 with pCP20 (Datsenko K A, et al. 2000. Proc Natl Acad Sci USA 97:6640-6645), which encodes the FLP recombinase, and transformants were selected on LB amp at 30° C. Deletion of the kan cassette was verified using PCR with primers BA2582 and BA2583, as well as screened for loss of pCP20, creating ASD200.

Construction SPI1 SPI2 fra triple mutant: Lambda Red mutagenesis was used to create a fraRBDAE island mutant (STM14_4332-STM14_4328), CS1005, using the protocol described above. Briefly, oligonucleotides BA2515 and BA2538 were used to amplify the kan cassette from pKD4 using Taq DNA polymerase (NEB). The PCR product was electroporated into 14028+pKD46 and transformants were selected on LB kan at 37° C. to create CS1005. The insertion of the kan cassette was verified by PCR using BA1922 (K1) and BA2888. The resulting fra4::kan island mutation was transduced into the ΔSPI1 ΔSPI2. strain ASD200 using the phage P22HTint, creating ASD201.

Animals: Swiss Webster mice were Obtained from Taconic Farms. CBA/j mice were obtained from Jackson Laboratories. Germ-free C57BL/6 and Swiss Webster mice were bred at the OSU germ-free facility. All mice were females between 6 and 10 weeks of age. All bacterial inocula were grown with shaking at 37° C. overnight, resuspended in water, and administered by the intragastric route in a volume of 200 μl.

RNA isolation from cecal tissues: Cecal samples were removed from mice and a portion was placed in RNAlater and stored at 4° C. until further processed for total RNA. Total RNA was isolated from cecal tissues using a TRI reagent (Sigma) and 1-bromo-3-chloropropane. Tissue was homogenized with TRI reagent in the TIssuelyzer LT (Qiagen).

Quantitative real-time PCR: Transcript levels of murine genes, IFNγ, TNFα, and GAPDH were determined from RNA isolated from cecal tissues. For quantitative analysis of mRNA, cDNA was made from 1 μg of total RNA in a 20 μl reaction using Taqman reverse transcription reagent (Applied Biosystems) using the oligo (d)T protocol. The cDNA reaction was diluted to a total volume of 100 μl and 2 μl of cDNA was used for the real time reaction. Real time PCR was done using the iQ Syber Green Mastermix (Bio-Rad) in the CFX96 Real-Time System (BioRad) with the CFX Manager software (BioRad). Relative quantitative expression of IFNγ and TNFα were done using the Livak method (PMID:11846609). The gene expression of each sample was normalized to GAPDH, then the target cytokine expression was calculated relative to the average target cytokine expression in five mock control mice.

Histopathology: Cecal samples were removed from mice and a portion was fixed in formalin. Samples were sent to the Comparative Pathology and Mouse Phenotyping Shared Resource at the Ohio State University College of Veterinary Medicine where the sample was embedded in paraffin, sectioned and stained with hematoxylin and eosin. A veterinary pathologist scored blinded samples for inflammation.

Animal assurance: All animal work was performed in accordance with the protocols approved by our Institutional Animal Care and Use Committee (OSU 2009A0035). The IACUC ensures compliance of this protocol with the U.S Animal Welfare Act, Guide for Care and Use of Laboratory Animals and Public Health Service Policy on Humane Care and Use of Laboratory Animals.

TABLE 4
Strains and Plasmids

| | Genotype | Reference |
|---|---|---|
| Strain | | |
| Escherichia coli | E. coli Nissle, serotype O6:K5:H1 | NISSLE A. 1959. Medizinische |
| Nissle 1917 | | 4: 1017-1022 |
| 14028 | wild-type Salmonella enterica serovar | American Type Culture Collection |
| | Typhimurium | |
| ASD100 | 14028 Δ(ssrB-ssaU)1::kan | Lambda red mutation of SPI2 using |
| | | primers BA2558 and BA2559. |
| ASD199 | 14028 Δ(avrA-invH)1 Δ(ssrB- | Δ(ssrB-ssaU)1::kan mutation from |
| | ssaU)1::kan | ASD100 transduced into YD039. |
| ASD200 | 14028 Δ(avrA-invH)1 Δ(ssrB-ssaU)1 | Kan cassette in ASD199 was |
| | | flipped out using pCP20. |
| ASD201 | 14028 Δ(avrA-invH)1 Δ(ssrB-ssaU)1 | Δ(fraR-fraBDAE)4::kan mutation |
| | Δ(fraR-fraBDAE)4::kan | from CS1005 was transduced into |
| | | ASD200. |
| ASD9000 | E. coli Nissle 1917 + pWSK29 (amp^{r}) | E. coli Nissle 1917 electroporated |
| | | with empty vector pWSK29. |
| ASD9010 | E. coli Nissle 1917 + pASD5006 | E. coli Nissle 1917 electroporated |
| | (amp^{r}) | with pASD5006. |
| CS1005 | 14028 Δ(fraR-fraBDAE)4::kan | Lambda red mutation of fra island |
| | | using primers BA2515 and |
| | | BA2538. |
| JLD1214 | 14028 IG(pagC-STM14_1502)::cam | Ali MM, et al. 2014. PLoS Pathog |
| | | 10: e1004209 |
| JM110 | rpsL thr leu thi-1 lacY galK galT ara | Stratagene |
| | tonA tsx dam dan supE44 Δ(lac- | |
| | proAB) | |
| YD039 | 14028 Δ(avrA-invH)1 | Teplitski M, et al. 2006. |
| | | Microbiology (Reading, Engl) |
| | | 152: 3411-3424 |
| Plasmids | | |
| pASD5006 | pWSK29 fraR-fraBDAE (amp^{r}) | Ali MM, et al. 2014. PLoS Pathog |
| | | 10: e1004209 |
| pWSK29 | pSC101 cloning vector (amp^{r}) | Ali MM, et al. 2014. PLoS Pathog |
| | | 10: e1004209 |

TABLE 5
Oligonucleotides

| Oligo- | | |
|---|---|---|
| nucleotide | Sequence | Description |
| BA1922 | CAGTCATAGCCGAAT | Kanamycin cassette |
| | AGCCT | insertion verification |
| | (SEQ ID NO: 17) | primer |
| BA2515 | GCCTGCATGATTAAT | Lambda red mutagenic |
| | ACGTACTGAAATAAC | reverse primer for |
| | TCTGGATCAGCATAT | STM14_4328 with P2 |
| | GAATATCCTCCTTAG | priming site |
| | (SEQ ID NO: 18) | |
| BA2538 | ATGGATACAAATGAT | Lambda red mutagenic |
| | CGAGCAACCCGACAG | forward primer for |
| | TAAAAGCGCCGTGTA | STM14_4332 with P1 |
| | GGCTGGAGCTGCTTC | priming site |
| | (SEQ ID NO: 19) | |
| BA2558 | ACGCCCCTGGTTAAT | Lambda red mutagenic |
| | ACTCTATTAACCTCA | forward primer with |
| | TTCTTCGGGCGTGTA | homology to ssrB with |
| | GGCTGGAGCTGCTTC | P1 priming site |
| | (SEQ ID NO: 20) | |
| BA2559 | CCAAAAGCATTTATG | Lambda red mutagenic |
| | GTGTTTCGGTAGAAT | reverse primer with |
| | GCGCATAATCCATAT | homology to ssaU with |
| | GAATATCCTCCTTAG | P2 priming site |
| | (SEQ ID NO: 21) | |
| BA2582 | AAATAAGGGGATTCT | Reverse primer for |
| | ACTATATCATGATCA | confirmation of SPI2 |
| | (SEQ ID NO: 22) | deletion |
| BA2583 | GCCAGGCTAAAAGCG | forward primer for |
| | ATTATTTTCAGTCTC | confirmation of SPI2 |
| | (SEQ ID NO: 23) | deletion |
| B2888 | GGATCCGCTTCGATA | Forward primer for |
| | CCTGAGTGGCAAAGT | verification of fra |
| | GTGCG | island mutation |
| | (SEQ ID NO: 24) | with K1. |

Discussion

The fra locus was identified in a genetic screen for Salmonella genes that are differentially required for fitness in germ-free mice colonized, or not, with the commensal organism Enterobacter cloacae (Ali M M, et al. 2014. PLoS Pathog 10:e1004209). Further experimentation revealed that a fraB mutation was severely attenuated in its ability to compete with wild-type Salmonella in four mouse models of inflammation: germ-free, germ-free colonized with human fecal microbiota, strep-treated, and IL-10 knockout. Interestingly, the fraB mutation was not attenuated in conventional mice that fail to become inflamed from Salmonella infection. It was also determined that a fraB mutation has no phenotype in inflamed mice if the competition experiment is performed in a Salmonella genetic background lacking SPI1 and SPI2 or ttrA. These results were interpreted to mean that SPI1 and SPI2 are required for Salmonella to induce inflammation (in models that are permissive), the inflammation is required to create tetrathionate and to kill microbes that would otherwise compete for F-Asn, and that ttrA is required for Salmonella to take advantage of the presence of F-Asn through tetrathionate respiration (Ali M M, et al. 2014. PLoS Pathog 10:e1004209). This model gave rise to the idea that adding the fra locus to probiotic species, such as E. coli Nissle 1917, could give them the ability to compete with Salmonella for a critical nutrient source and thus prevent infection. Since then it was discovered that the fraB phenotype is primarily due to the accumulation of a toxic metabolite during growth on F-Asn rather than F-Asn being a particularly important nutrient source. Despite this, there seemed to be some fra-dependence with regard to the ability of the Salmonella SRI1 SPI2 mutant to compete with wild-type Salmonella, especially in CBA/J mice. It appeared that protection was fra-dependent because the SPI1 SPI2 double mutant, but not the SPI1 SPI2 fra triple mutant, was significantly different than sham. However, the double mutant is not statistically different than the triple mutant. Furthermore, the Nissle strain modified to encode the fra locus was altered in its ability to kill germ-free C57BL/6 mice and in its ability to protect germ-free Swiss Webster mice against Salmonella infection, compared to the original Nissle strain. These results suggest that F-Asn is a significant nutrient source in some situations, but definitely not the only nutrient source available to E. coli and Salmonella in the inflamed intestine.

The enhanced virulence of the Nissle+fra strain indicates that it will not make a commercially viable probiotic, although this does not rule out the possibility that adding fra to a different probiotic organism, such as Lactobacillus or Bifidobacterium, might still work. However, the Salmonella SPI1 SPI2 mutant looks promising. This strain was included in the study to determine what would happen if we continued adding Salmonella-specific nutrient acquisition loci to Nissle, essentially creating an avirulent Salmonella. Unlike Nissle, the Salmonella SPI1 SPI2 mutant can compete with wild-type Salmonella for all nutrient sources rather than for a subset. Currently, a cya cip mutant of Salmonella is used as a live attenuated vaccine strain in agriculture (Curtiss R 3rd, et al. 1987. Infect Immun 55:3035; Hassan J O, et al. 1991. Research in Microbiologoy 142:109; Kelly S M, et al. 1992, Infect Immun 60:4881-4890). This strain is metabolically attenuated so it cannot compete metabolically with wild-type Salmonella, but instead creates a lasting immune response against a single serovar. The use of a Salmonella SRI1 SPI2 mutant as a probiotic takes a different approach in which the strain is metabolically competent, so it should be able to compete effectively against hundreds of serovars of Salmonella. There is precedent for this approach in the literature. A non-toxigenic Clostridium difficile can compete with wild-type C. difficile to resolve infection and prevent recurrence (Gerding D N, et al. 2015. JAMA 313:1719-1727). The Salmonella SPI1 SPI2 mutant was most effective in protecting germ-free mice from wild-type Salmonella. This suggests that this strain might be particularly effective in preventing Salmonella colonization of neonatal agricultural animals such as newly hatched poultry or swine.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating or preventing Salmonella-induced gastroenteritis in a subject, comprising administering to the subject a probiotic composition comprising an avirulent but metabolically competent Salmonella bacterium in an amount effective to compete with Salmonella for available nutrients.

2. The method of claim 1, wherein the Salmonella bacterium has one or more inactivating mutations or deletions of the genes within its Salmonella Pathogenicity Island 1 (SPI1) locus, Salmonella Pathogenicity Island 2 (SPI2) locus, or a combination thereof.

3. The method of claim 2, wherein the entire SPI1 locus, SPI2 locus, or a combination thereof, has been deleted in the Salmonella bacterium.

4. The method of claim 2, wherein the genes are selected from the group consisting of sicA, sicP, invA, invB, invC, invI, invJ, invE, invG, invH, orgA, orgB, orgC, prgH, prgK, prgI, prgJ, spaM, spaN, spaO, spaP, spaQ, spaR, spaS, sipA, sipD, sipB, sipC, sseC, sseD, sseB, ssaU, ssaT, ssaS, ssaR, ssaQ, ssaP, ssaO, ssaG, ssaJ, ssaC, ssaV, ssaN, spiC, ssaL, ssaM, ssaK, ssaI, ssaH, ssaG, ssaB, ssaC, ssaD, ssaE, sscA, sscB, sseA, sseE, sseG, sseF, hilA, invF, hilC, hilD, iagB, sprA, sprB, ssrA, ssrB, sptP, avrA, sicP, and iacP.

5. The method of claim 2, wherein the Salmonella bacterium has reduced expression or activity of type 3 secretion system (T3 SS) effector proteins not present in the SPI1 or SPI2 locus.

6. The method of claim 5, wherein the T3 SS effector proteins are encoded by genes selected from the group consisting of sopA, sopB/sigD, sopE, sopE2, srgE, slrP, sopD, sspH1, steA, steB, gogB, pipB, pipB2, sifA, sifB, sopD2, sseI/srfH/gtgB, sseJ, sseK1, sseK2, sseK3, sseL, sspH2, steC, spvB, spvC, spvD, cigR, gtgA, gtgE, pipB2, srfJ, steD, and steE.

7. The method of claim 1, wherein the probiotic composition comprises 10⁷ to 10⁹ colony-forming units (CFU) of the Salmonella bacterium.

8. The method of claim 1, wherein the Salmonella bacterium is food-grade bacterium.

9. The method of claim 1, wherein the Salmonella bacterium is lyophilized.

10. The method of claim 1, wherein the Salmonella bacterium is encapsulated in a gel matrix.


SEQUENCE LISTING (24 sequences)

SEQ ID NO: 1 - DNA, 1320 bp, Salmonella bacterium
atgttttggacggaattatgttttatccttgtggccctgatgataggcgccaggatcggcggcgtatttttagggatggtcggcgggttaggcgtcggcgtgatggtttttatttttggcctgacgccttctacgccaccgattgatgttattctgattattctttctgttgtcctggcggccgcttctttacaggcctccggcgggctggatttactggtcaaactggcggaaaaaattctgcgtcgccacccgcgttacattacgttattagcgccgtttatctgttatatcttcacttttatgtcaggaacggggcatgtcgtttatagcttgctaccggttatttctgaagtcgcacgggattcaggtattcgaccggaacgtcctttatctatttccgttatcgcatcgcaacaggcgatcaccgccagtcctatatctgccgccatggcggcgatgattggtttaatggcgccgttgggcgtctctatttcaaccattatgatgatttgcgtgcccgccacgttaatcggcgtagcgatgggggcaatagcgacctttaataaaggaaaagagttaaaagacgatccggaatatcaacgtcggcttgctgaagggttaattaaacctgcgcagaaagaaagtaaaaatacggtggtcacttcgcgcgccaaattgtcggtggcgttatttctgaccagtgcgatcgttatcgttctgttaggactgattccggcgctgcggcccatggtggaaacagcgaaagggctacaaccgctttcgatgtccgccgctatccagattacgatgctctcttttgcctgcctgattgtgttgttatgccgaccgcaggtcgatcaaattatcagcggtacggtatttcgggcgggcgcgctggcgattgtctgcgccttcggcctggcctggatgagtgagacgttcgtgaatggtcatatcgcgttgattaaggcagaagtgcaaactctattgcaacagcatacctggcttatcgccattatgatgttttttgtgtccgctatggtcagcagccaggcggcaacgacgttaattctgttgccgctggggctggcgttagggttgcccgcttatgcattaatcggctcctggcctgccgttaacggctatttctttattccggtggcggggcagtgtctggcggcgctggcgtttgacgataccggtacgacgcgtattggcaaatatgtgcttaaccatagttttatgcgtccgggattagttaacgtgattgtctcggtcattgtcgggctgttaataggaaaaatggttctggcctga

SEQ ID NO: 2 - protein, 439 aa, Salmonella bacterium
MFWTELCFILVALMIGARIGGVFLGMVGGLGVGVMVFIFGLTPSTPPIDVILIILSVVLAAASLQASGGLDLLVKLAEKILRRHPRYITLLAPFICYIFTFMSGTGHVVYSLLPVISEVARDSGIRPERPLSISVIASQQAITASPISAAMAAMIGLMAPLGVSISTIMMICVPATLIGVAMGAIATFNKGKELKDDPEYQRRLAEGLIKPAQKESKNTVVTSRAKLSVALFLTSAIVIVLLGLIPALRPMVETAKGLQPLSMSAAIQITMLSFACLIVLLCRPQVDQIISGTVFRAGALAIVCAFGLAWMSETFVNGHIALIKAEVQTLLQQHTWLIAIMMFFVSAMVSSQAATTLILLPLGLALGLPAYALIGSWPAVNGYFFIPVAGQCLAALAFDDTGTTRIGKYVLNHSFMRPGLVNVIVSVIVGLLIGKMVLA

SEQ ID NO: 3 - DNA, 978 bp, Salmonella bacterium
atgatgggtatgaaagagacagttagcaatattgtgaccagccaggcagagaaaggaggcgttaaacacgtctattacgtggcgtgcggcggttcttatgcggcgttctatccggcgaaagcatttttagaaaaagaagcgaaagcgttgactgtcggtctgtataacagcggagaatttattaacaacccgccggtagcgctgggagaaaatgccgttgtggttgtcgcctcccacaaaggtaatacgccagagacaattaaagcggctgaaatcgcccgtcagcacggcgcgccggtcattggtttaacctggataatggattcaccgttggtggcgcattgcgactatgtggaaacgtacacgtttggcgacggtaaagatattgccggagagaaaacgatgaaaggcctgctgagtgcggtcgaactgctccagcagacggaagggtatgcgcactacgacgattttcaggatggcgtcagcaaaatcaaccgtatcgtctggcgcgcttgcgagcaggtagcggagcgtgcgcaggcgttcgcgcaggaatataaagacgataaagtcatttataccgtcgccagcggcgcgggctatggcgcagcctacctacagagcatctgcatctttatggaaatgcaatggatacattccgcctgtattcatagcggtgagtttttccacgggccgtttgaaattaccgatgcgaatacgcctttcttcttccagttttccgagggcaatacgcgggcggtggatgaacgcgcgttaaacttcctgaaaaaatatggccgccggattgaagttgtcgatgcgaaagaactggggctatcgaccattaaaaccacggttattgattactttaaccactctctctttaataacgtttatcccgtttacaatcgggcgttagctgaggcgcgtcagcatccgttaacgacgcgccgctatatgtggaaagtggaatattaa

SEQ ID NO: 4 - protein, 325 aa, Salmonella bacterium
MMGMKETVSNIVTSQAEKGGVKHVYYVACGGSYAAFYPAKAFLEKEAKALTVGLYNSGEFINNPPVALGENAVVVVASHKGNTPETIKAAEIARQHGAPVIGLTWIMDSPLVAHCDYVETYTFGDGKDIAGEKTMKGLLSAVELLQQTEGYAHYDDFQDGVSKINRIVWRACEQVAERAQAFAQEYKDDKVIYTVASGAGYGAAYLQSICIFMEMQWIHSACIHSGEFFHGPFEITDANTPFFFQFSEGNTRAVDERALNFLKKYGRRIEVVDAKELGLSTIKTTVIDYFNHSLFNNVYPVYNRALAEARQHPLTTRRYMWKVEY

SEQ ID NO: 5 - DNA, 846 bp, Salmonella bacterium
atgagcatcagcgtattgggtattggcgacaacgttgtcgataaatacctgcattccggcatcatgtaccccggcggtaatgcattaaattttgctgtctatgcgaaattagcagacatccccagcgcgtttatgggggcgtttggcaatgacgacgccgcgcagcacgtacaggatgtattacaccagctacagatagacatctctcacagccgccattataccggcgaaaatgggtatgcctgtatccgtctctcgcatggcgatcggcaatttgtcgccagcaacaaaaacggcgtattgcgggaacatccttttagtctgtctgacgacgatcttcgctatatatcacaatttaccttagtccattccagtattaacggccacctggaatcggaactggagaaaattaaacaacaaaccgtcttactctcttttgatttttccgggcgcggtacagacgactattttgaaaaggtatgcccgtgggtagattacggatttatctcctgtagcgggttatcgccagatgaaatcaaagtaaaactcaataaactttatcgttatggctgtcggcatattattgccacctgcgggcatgaaaaagtttattatttttccggcgcggattatctggagtggcaacctgcttatatcgaacctgtcgatacgctgggcgcaggcgacgccttcttaaccggttttttgctttccattttgcaatcgggtatggcggaacccgataaagaaagcgtgttacgcgccatgcggcagggcgggaaatcggcggcgcaggtgttatctcattacggcgcatttggttttggtaaaccgtttgcacaatag

SEQ ID NO: 6 - protein, 281 aa, Salmonella bacterium
MSISVLGIGDNVVDKYLHSGIMYPGGNALNFAVYAKLADIPSAFMGAFGNDDAAQHVQDVLHQLQIDISHSRHYTGENGYACIRLSHGDRQFVASNKNGVLREHPFSLSDDDLRYISQFTLVHSSINGHLESELEKIKQQTVLLSFDFSGRGTDDYFEKVCPWVDYGFISCSGLSPDEIKVKLNKLYRYGCRHIIATCGHEKVYYFSGADYLEWQPAYIEPVDTLGAGDAFLTGFLLSILQSGMAEPDKESVLRAMRQGGKSAAQVLSHYGAFGFGKPFAQ

SEQ ID NO: 7 - DNA, 1044 bp, Salmonella bacterium
atgaaaattagagttttcatggccaccgtgttgctgctcatcagccactgtgtatttagcacaacgtcactaccgcatattgttattctcgcgacaggtggtactatcgccgggacggcagccaataatacgcaaaccgccggatataaatctggtgaacttggcgtgcaaacattaataaatgccgtgccggaaatgaataatatcgctcgcgttgacggcgagcaggtggcgaatattggtagcgaaaatatgaccagcgatatcatcctgaaactttcacagaaggtgaatgcgttattggcgcgggacgatgttgacggtgtggttattactcatggcactgacacgctcgatgaaaccgcctactttcttaatttgaccgtgaaaagcgacaaaccggtggtgtttaccgctgcaatgcggcccgcgtcggcaatcagcgccgatggcgcaatgaacctgctggaagcggtcacggtggctgctgacccgaatgcgaagggacgcggtgtgatggtggttttaaacgatcgtattggttcggcgcgctttgtgacgaaaactaatgccacgactctggatacctttaaagcgccggaagagggctatctgggggtcatcgttaatggtcagccacagttcgaaacgcgggtggaaaaaattcataccctgcgatctgtttttgacgtacgtaatatcaaaaaattacccaatgtggtgattatttacggctatcaggacgacccggaatatatgtatgatgcggcgatcgcccatcacgcggacggtattatttatgccggaaccggcgcaggttcggtctcggtacgcagcgacgcggggattaaaaaagcggagaaagccgggattatcgtggtgcgcgcttcccgcaccggaaacggcgtcgtaccgttggataaagggcagccagggctggtgtctgactcgctcaacccggcgaaggcgcgagtcttgctgatgacggcattaactcagacgcgtaatccggaactgatccagagttatttcagtacgtattaa

SEQ ID NO: 8 - protein, 347 aa, Salmonella bacterium
MKIRVFMATVLLLISHCVFSTTSLPHIVILATGGTIAGTAANNTQTAGYKSGELGVQTLINAVPEMNNIARVDGEQVANIGSENMTSDIILKLSQKVNALLARDDVDGVVITHGTDTLDETAYFLNLTVKSDKPVVFTAAMRPASAISADGAMNLLEAVTVAADPNAKGRGVMVVLNDRIGSARFVTKTNATTLDTFKAPEEGYLGVIVNGQPQFETRVEKIHTLRSVFDVRNIKKLPNVVIIYGYQDDPEYMYDAAIAHHADGIIYAGTGAGSVSVRSDAGIKKAEKAGIIVVRASRTGNGVVPLDKGQPGLVSDSLNPAKARVLLMTALTQTRNPELIQSYFSTY

SEQ ID NO: 9 - DNA, 720 bp, Salmonella bacterium
atgatcgagcaacccgacagtaaaagcgccaaaccgctttataagcagcttgaagccgccttaaaagaggctattgcgcgtggagagtataaaccaggccagcagatcccgacggaaaatgaactgagcgtgcgctggcaggtgagcagggtcacggtccgtaaggcgctggatgcgctgacgcgtgaaaatttgctgacccgtgtctccggcaaaggcacctttgtctctggtgagaaatttcagcgcagcatgaccggcatcatgagtttcagcgagttatgccagtcccagggacgtcgcccggggtcacgcaccatcaaatccgtttttgaatcggtagacgatgagacaaaagcgttactgaatatgaacgatggcgaaaaagcggtcgtcattgaacgtatccgctatgccgacgatgtggcggtatcgctggaaaccgtacatcttcccccacgttttgcgtttttgctggacgaagatcttaataatcactctttgtatgaatgcttacgcgagaaataccatttatggtttacccactcccgtaagatgatcgaactggtttatgccagctttgaagtcgcccattatcttggcgtcaacgagggttatccgctgatcctgataaaaagtgaaatgattgataacaaaggagaactctcctgcgtttcgcaacagttgattgtcggcgataaaatacggtttaccgtatga

SEQ ID NO: 10 - protein, 239 aa, Salmonella bacterium
MIEQPDSKSAKPLYKQLEAALKEAIARGEYKPGQQIPTENELSVRWQVSRVTVRKALDALTRENLLTRVSGKGTFVSGEKFQRSMTGIMSFSELCQSQGRRPGSRTIKSVFESVDDETKALLNMNDGEKAVVIERIRYADDVAVSLETVHLPPRFAFLLDEDLNNHSLYECLREKYHLWFTHSRKMIELVYASFEVAHYLGVNEGYPLILIKSEMIDNKGELSCVSQQLIVGDKIRFTV

SEQ ID NO: 11 - DNA, 61 bp, Artificial Sequence
  synthetic construct
cttctttaccagtgacacgtacctgcctgtcttttctcttgtgtaggctggagctgcttcg

SEQ ID NO: 12 - DNA, 58 bp, Artificial Sequence
  synthetic construct
cgaaggcggtcacaaaatcttgatgacattgtgattaacatatgaatatcctccttag

SEQ ID NO: 13 - DNA, 28 bp, Artificial Sequence
  synthetic construct
cgcagaatctatccgtccgacaacgaac

SEQ ID NO: 14 - DNA, 30 bp, Artificial Sequence
  synthetic construct
gcaggttaaggctctccgtaaaggccaatc

SEQ ID NO: 15 - DNA, 60 bp, Artificial Sequence
  synthetic construct
cctgatgtaattaatattccactttccacatatagcggcgcatatgaatatcctccttag

SEQ ID NO: 16 - DNA, 60 bp, Artificial Sequence
  synthetic construct
agaggaaagcatgatgggtatgaaagagacagttagcaatgtgtaggctggagctgcttc

SEQ ID NO: 17 - DNA, 20 bp, Artificial Sequence
  synthetic construct
cagtcatagccgaatagcct

SEQ ID NO: 18 - DNA, 60 bp, Artificial Sequence
  synthetic construct
gcctgcatgattaatacgtactgaaataactctggatcagcatatgaatatcctccttag

SEQ ID NO: 19 - DNA, 60 bp, Artificial Sequence
  synthetic construct
atggatacaaatgatcgagcaacccgacagtaaaagcgccgtgtaggctggagctgcttc

SEQ ID NO: 20 - DNA, 60 bp, Artificial Sequence
  synthetic construct
acgcccctggttaatactctattaacctcattcttcgggcgtgtaggctggagctgcttc

SEQ ID NO: 21 - DNA, 60 bp, Artificial Sequence
  synthetic construct
ccaaaagcatttatggtgtttcggtagaatgcgcataatccatatgaatatcctccttag

SEQ ID NO: 22 - DNA, 30 bp, Artificial Sequence
  synthetic construct
aaataaggggattctactatatcatgatca

SEQ ID NO: 23 - DNA, 30 bp, Artificial Sequence
  synthetic construct
gccaggctaaaagcgattattttcagtctc

SEQ ID NO: 24 - DNA, 35 bp, Artificial Sequence
  synthetic construct
ggatccgcttcgatacctgagtggcaaagtgtgcg